(19) United States
(12) Patent Application Publication
(10) Pub. No.: US 2002/0019342 A1
(43) Pub. Date: Feb. 14, 2002

(54) In vitro modification of glycosylation patterns of recombinant glycopeptides

(76) Inventor: Robert Bayer, San Diego, CA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/855,320

(22) Filed: May 14, 2001

Related U.S. Application Data
(60) Provisional application No. 60/203,851, filed on May 12, 2000.

(51) Int. Cl. A61K 38/16; C12Q 1/54

(52) U.S. Cl. 514/8; 435/14

(57) ABSTRACT

This invention provides methods for modifying glycosylation patterns of glycopeptides, including recombinantly produced glycopeptides. Also provided are glycopeptide compositions in which the glycopeptides have a uniform glycosylation pattern.

### CROSS-REFERENCES TO RELATED APPLICATIONS

This application claims the benefit of prior U.S. provisional application number 60/203,851, filed May 12, 2000.

### BACKGROUND OF THE INVENTION

1. Field of the Invention

This invention pertains to the field of methods for modifying the glycosylation pattern on glycopeptides.

2. Background

A. Protein Glycosylation

The biological activity of many glycoproteins is highly dependent upon the presence or absence of particular oligosaccharide structures attached to the glycoprotein. Improperly glycosylated glycoproteins are implicated in cancer, infectious diseases and inflammation (Dennis et al., BioEssays 21: 412-421 (1999)). Moreover, the glyosylation pattern of a therapeutic glycoprotein can affect numerous aspects of the therapeutic efficacy such as solubility, resistance to proteolytic attack and thermal inactivation, immunogenicity, half-life, bioactivity, and stability (see, e.g., Rotondaro et al., Mol. Biotechnol. 11: 117-128 (1999); Lis et al., Eur. J. Biochem. 218: 1-27 (1993); Ono et al., Eur. J. Cancer 30A (Suppl. 3), S7-S11(1994); and Hotchkiss et al., Thromb. Haemost. 60: 255-261 (1988)). Regulatory approval of therapeutic glycoproteins also requires that the glycosylation be homogenous and consistent from batch to batch.

Glycosylation is a complex post-translational modification that is highly cell dependent. Following translation, proteins are transported into the endoplasmic reticulum (ER), glycosylated and sent to the Golgi for further processing. The resulting glycoproteins are subsequently targeted to various organelles, become membrane components, or they are secreted into the periplasm.

During glycosylation, either N-linked or O-linked glycoproteins are formed. N-glycosylation is a highly conserved metabolic process, which in eukaryotes is essential for viability. N-linked glycosylation is also implicated in development and homeostasis; N-linked glycoproteins constitute the majority of cell-surface proteins and secreted proteins, which are highly regulated during growth and development (Dennis et al., Science 236: 582-585 (1987)). N-glycosylation is also believed to be related to morphogenesis, growth, differentiation and apoptosis.

In eukaryotes, N-linked glycosylation occurs on the asparagine of the consensus sequence Asn-Xₐₐ-Ser/Thr, in which Xₐₐ is any amino acid except proline (Komfeld et al., Ann Rev Biochem 54: 631-664 (1985); Kukuruzinska et al., Proc. Natl. Acad. Sci. USA 84: 2145-2149 (1987); Herscovics et al., FASEB J 7:540-550 (1993); and Orlean, Saccharomyces Vol. 3 (1996)). O-linked glycosylation also takes place at serine or threonine residues (Tanner et al., Biochim. Biophys. Acta. 906: 81-91 (1987); and Hounsell et al., Glycoconj. J. 13: 19-26 (1996)). Other glycosylation patterns are formed by linking glycosylphosphatidylinositol to the carboxyl-terminal carboxyl group of the protein (Takeda et al., Trends Biochem. Sci. 20: 367-371 (1995); and Udenfriend et al., Ann. Rev. Biochem. 64: 593-591 (1995)).

The biosynthesis of N-linked glycoproteins is initiated with the dolichol pathway in the endoplasmic reticulum (Burda, P., et al., Biochimica et Biophysica Acta 1426: 239-257 (1999); Komfeld et al., Ann. Rev. Biochem. 54: 631-664 (1985); Kukuruzinska et al., Ann. Rev. Biochem. 56: 915-944 (1987); Herscovics et al., FASEB J. 7: 540-550 (1993)). At the heart of the dolichol pathway is the synthesis of an oligosaccharide linked to a polyisoprenol carrier lipid. The oligosaccharide, GlcNAc₂Man₉Glc₃, is assembled through the glycosyl-transferase catalyzed, stepwise addition of monosaccharides. The dolichol pathway is highly conserved between yeast and mammals.

After the assembly of the dolichol-oligosaccharide conjugate, the oligosaccharide is transferred from this conjugate to an asparagine residue of the protein consensus sequence. The transfer of the oligosaccharide is catalyzed by the multi-subunit enzyme oligosaccharyltransferase (Karaoglu et al., Cold Spring Harbor Symposia on Quantitative Biology LX: 83-92 (1995b); and Silberstein et al., FASEB J. 10:849-858 (1996). Subsequent to the transfer of the oligosaccharide to the protein, a series of reactions, which shorten the oligosaccharide occur. The reactions are catalyzed by glucosidases I and II and α-mannosidase (Kilker et al., J. Biol. Chem., 256: 5299-5303 (1981); Saunier et al., J. Biol. Chem. 257: 14155-14161 (1982); and Byrd et al., J. Biol. Chem. 257:14657-14666 (1982)).

Following the synthesis and processing of the N-linked glycoprotein in the endoplasmic reticulum, the glycoprotein is transported to the Golgi, where various processing steps result in the formation of the mature N-linked oligosaccharide structures. Although the dolichol pathway is highly conserved in eukaryotes, the mature N-linked glycoproteins produced in the Golgi exhibit significant structural variation across the species. For example, yeast glycoproteins include oligosaccharide structures that consist of a high mannose core of 9-13 mannose residues, or extended branched mannan outer chains consisting of up to 200 residues (Ballou, et al., Dev. Biol. 166: 363-379 (1992); Trimble et al., Glycobiology 2: 57-75 (1992)). In higher eukaryotes, the N-linked oligosaccharides are typically high mannose, complex and mixed types of structures that vary significantly from those produced in yeast (Komfeld et al., Ann. Rev. Biochem. 54: 631-664 (1985)). Moreover, in yeast, a single α-1,2-mannose is removed from the central arm of the oligosaccharide, in higher eukaryotes, the removal of mannose involves the action of several mannosidases to generate a GlcNAc₂Man₅ structure (Kukuruzinska et al., Crit Rev Oral Biol Med. 9(4): 415-448 (1998)). The branching of complex oligosaccharides occurs after the trimming of the oligosaccharide to the GlcNAc₂Man₅ structure. Branched structures, e.g. bi-, tri-, and tetra-antennary, are synthesized by the GlcNAc transferase-catalyzed addition of GlcNAc to regions of the oligosaccharide residue. Subsequent to their formation, the antennary structures are terminated with different sugars including Gal, GalNAc, GlcNAc, Fuc and sialic acid residues.

Similar to N-glycosylation, O-glycosylation is also markedly different between mammals and yeast. At the initiation of O-glycosylation, mammalian cells add a GalNAc residue directly to Ser or Thr using UDP-GalNAc as a glycosyl donor. The saccharide unit is elongated by adding Gal, GlcNAc, Fuc and NeuNAc. In contrast to mammalian cells, lower eukaryotes, e.g., yeast and other fingi, add a mannose to Ser or Thr using Man-P-dolichol as a glycosyl donor. The saccharides are elongated by adding Man and/or Gal. See, generally, Gemmill et al., Biochim. Biophys Acta 1426: 227-237 (1999).

Efforts to elucidate the biological mechanism of protein glycosylation and the glycosylation patterns of glycoproteins have been aided by a number of analytical techniques. For example, N-linked oligosaccharides of recombinant aspartic protease were characterized using a combination of mass spectrometric, 2D chromatographic, chemical and enzymatic methods (Montesino et al., Glycobiology 9: 1037-1043 (1999)). The same workers have also reported the characterization of oligosaccharides enzymatically released from purified glycoproteins using fluorescent-labeled derivatives of the released oligosaccharides in combination with fluorophore-assisted carbohydrate electrophoresis (FACE) (Montesino et al., Protein Expression and Purification 14: 197-207 (1998)).

Cloned endo- and exo-glycosidases are standardly used to release monosaccharides and N-glycans from glycoproteins. The endoglycosidases allow discrimination between N-linked and O-linked glycans and between classes of N-glycans. Methods of separating glycoproteins on separated glycans have also become progressively more sophisticated and selective. Methods of separating mixtures of glycoproteins and cleaved glycans have also continued to improve and techniques such as high pH anion exchange chromatography (HPAEC) are routinely used for the separation of individual oligosaccharide isomers from a complex mixture of oligosaccharides. Recently, a large-scale organic solvent (acetone) precipitation-based method for isolating saccharides released from glycosaccharides was reported by Verostek et al. (Analyt. Biochem. 278: 111-122 (2000)). Many other methods of isolating and characterizing oligosaccharides released from glycoproteins are known in the art. See, generally, Fukuda et al., GLYCOBIOLOGY: A PRACTICAL APPROACH, Oxford University Press, New York 1993; and E. F. Hounsell (Ed.) GLYCOPROTEIN ANALYSIS IN BIOMEDICINE, Humana Press, Totowa, N.J., 1993.

B. Synthesis of Glycoproteins

Considerable effort has been directed towards the identification and optimization of new strategies for the preparation of saccharides and glycoproteins derived from these saccharides. Included amongst the many promising methods are the engineering of cellular hosts that produce glycoproteins having a desired glycosylation pattern, chemical synthesis, enzymatic synthesis, enzymatic remodeling of formed glycoproteins and methods that are hybrids of one or more of these techniques.

Cell host systems have been investigated in which glycoproteins of interest as pharmaceutical agents can be produced in commercially feasible quantities. In principle, mammalian, insect, yeast, fungal, plant or prokaryotic cell culture systems can be used for production of most therapeutic and other glycoproteins. In practice, however, a desired glycosylation pattern on a recombinantly produced protein is difficult to achieve. For example, bacteria do not N-glycosylate via the dolichol pathway, and yeast and make only oligomannose-type N-glycans, which are not generally found in humans. (see, e.g., Ailor et al. Glycobiology 1: 837-847 (2000)). Similarly, plant cells do not produce sialylated oligosaccharides, a common constituent of human glycoproteins (see, generally, Liu, Trends Biotechnol 10: 114-20 (1992); and Lerouge et al., Plant Mol. Biol. 38: 31-48 (1998)). As recently reviewed, none of the insect cell systems presently available for the production of recombinant mammalian glycoproteins will produce glycoproteins with the same glycans normally found when they are produced in mammals. Moreover, glycosylation patterns of recombinant glycoproteins frequently differ when they are produced under different cell culture conditions (Watson et al. Biotechnol. Prog. 10: 39-44 (1994); and Gawlitzek et al., Biotechnol. J. 42: 117-131 (1995)). It now appears that glycosylation patterns of recombinant glycoproteins can vary between glycoproteins produced under nominally identical cell culture conditions in two different bioreactors (Kunkel et al., Biotechnol. Prog. 2000:462-470 (2000)). Finally, in many bacterial systems, the recombinantly produced proteins are completely unglycosylated.

Heterogeneity in the glycosylation of a recombinantly produced glycoproteins arises because the cellular machinery (e.g., glycosyltransferases and glycosidases) may vary from species to species, cell to cell, or even from individual to individual. The substrates recognized by the various enzymes may be sufficiently different that glycosylation may not occur at some sites or may be vastly modified from that of the native protein. Glycosylation of recombinant proteins produced in heterologous eukaryotic hosts will often differ from the native protein. For example, yeast and insect expressed glycoproteins typically contain high mannose structures that are not commonly seen in humans.

An area of great interest is the design of host cells that have the glycosylation apparatus necessary to prepare properly glycosylated recombinant human glycoproteins. The Chinese hamster ovary (CHO) cell is a model cell system that has been particularly well studied, because CHO cells are equipped with a glycosylation machinery that is very similar to that found in the human (Jenkins et al., Nature Biotechnol. 14: 975-981 (1996)). In contrast to the many similarities between the glycosylation patterns of glycoproteins from human cells and those from CHO cells, an important distinction exists; glycoproteins produced by CHO cells carry only α-2,3-terminal sialic acid residues, whereas those produced by human cells include both α-2,3- and α-2,6-terminal sialic acid residues (Lee et al., J. Biol. Chem. 264: 13848-13855 (1989)).

Efforts to remedy the deficiencies of the glycosylation of a particular host cell have focused on engineering the cell to express one or more missing enzymes integral to the human glycosylation pathway. For example, Bragonzi et al. (Biochim. Biophys. Acta 1474: 273-282 (2000)) have produced a CHO cell that acts as a ‘universal host’ cell, having both α-2,3- and α-2,6-sialyltransferase activity. To produce the universal host, CHO cells were transfected with the gene encoding expression of α-2,6-sialyltransferase. The resulting host cells then underwent a second stable transfection of the genes encoding other proteins, including human interferon γ(IFN-γ). Proteins were recovered that were equipped with both α-2,3- and α-2,6-sialic acid residues. Moreover, in vivo pharmacokinetic data for IFN-γ demonstrate improved pharmacokinetics of the IFN-γ produced by the universal host, as compared to the IFN-γ secreted by regular CHO cells transfected with IFN-γ cDNA.

In addition to preparing properly glycosylated glycoproteins by engineering the host cell to include the necessary compliment of enzymes, efforts have been directed to the development of both de novo synthesis of glycoproteins and the in vitro enzymatic methods of tailoring the glycosylation of glycoproteins. Methods of synthesizing both O-linked and N-linked glycopeptides have been recently reviewed (Arsequell et al., Tetrahedron: Assymetry 8: 2839 (1997); and Arsequell et al., Tetrahedron: Assymetry 10: 2839 (1997), respectively)).

Two broad synthetic motifs are used to synthesize N-linked glycopeptides: the convergent approach; and the stepwise building block approach. The stepwise approach generally makes use of solid-phase peptide synthesis methodology, originating with a glycosyl asparagine intermediate. In the convergent approach, the peptide and the carbohydrate are assembled separately and the amide linkage between these two components is formed late in the synthesis. Although great advances have been made in recent years in both carbohydrate chemistry and the synthesis of glycoproteins, there are still substantial difficulties associated with chemical synthesis of glycoproteins, particularly with the formation of the ubiquitous β-1,2-cis-mannoside linkage found in mammalian oligosaccharides. Moreover, regio- and stereo-chemical obstacles must be resolved at each step of the de novo synthesis of a carbohydrate. Thus, this field of organic synthesis lags substantially behind the de novo synthesis of other biomolecules such as oligonucleotides and peptides.

In view of the difficulties associated with the chemical synthesis of carbohydrates, the use of enzymes to synthesize the carbohydrate portions of glycoproteins is a promising approach to preparing glycoproteins. Enzyme-based syntheses have the advantages of regioselectivity and stereoselectivity. Moreover, enzymatic syntheses can be performed using unprotected substrates. Three principal classes of enzymes are used in the synthesis of carbohydrates, glycosyltransferases (e.g., sialyltransferases, oligosaccharyltransferases, N-acetylglucosaminyltransferases), Glycoaminidases (e.g., PNGase F) and Glycosidases. The glycosidases are further classified as exoglycosidases (e.g., β-mannosidase, β-glucosidase), and endoglycosidases (e.g., Endo-A, Endo-M). Each of these classes of enzymes has been successfully used synthetically to prepare carbohydrates. For a general review, see, Crout et al., Curr. Opin. Chem. Biol. 2: 98-111 (1998) and Arsequell, supra.

Glycosyltransferases have been used to modify the oligosaccharide structures on glycoproteins. Glycosyltransferases have been shown to be very effective for producing specific products with good stereochemical and regiochemical control. Glycosyltransferases have been used to prepare oligosaccharides and to modify terminal N- and O-linked carbohydrate structures, particularly on glycoproteins produced in mammalian cells. For example, the terminal oligosaccharides have been completely sialylated and/or fucosylated to provide more consistent sugar structures, which improves glycoprotein pharmacodynamics and a variety of other biological properties. For example, β-1,4-galactosyltransferase was used to synthesize lactosamine, the first illustration of the utility of glycosyltransferases in the synthesis of carbohydrates (see, e.g., Wong et al., J. Org. Chem. 47: 5416-5418 (1982)). Moreover, numerous synthetic procedures have made use of α-sialyltransferases to transfer sialic acid from cytidine-5′-monophospho-N-acetylneuraminic acid to the 3-OH or 6-OH of galactose (see, e.g., Kevin et al., Chem. Eur. J. 2: 1359-1362 (1996)). For a discussion of recent advances in glycoconjugate synthesis for therapeutic use see, Koeller et al., Nature Biotechnology 18: 835-841 (2000).

Glycosidases normally catalyze the hydrolysis of a glycosidic bond, however, under appropriate conditions they can be used to form this linkage. Most glycosidases used for carbohydrate synthesis are exoglycosidases; the glycosyl transfer occurs at the non-reducing terminus of the substrate. The glycosidase takes up a glycosyl donor in a glycosylenzyme intermediate that is either intercepted by water to give the hydrolysis product, or by an acceptor, to give a new glycoside or oligosaccharide. An exemplary pathway using a exoglycoside is the synthesis of the core trisaccharide of all N-linked glycoproteins, including the notoriously difficult β-mannoside linkage, which was formed by the action of β-mannosidase (Singh et al., Chem. Commun. 993-994 (1996)).

Although their use is less common than that of the exoglycosidases, endoglycosidases have also been utilized to prepare carbohydrates. Methods based on the use of endoglycosidases have the advantage that an oligosaccharide, rather than a monosaccharide, is transferred. Oligosaccharride fragments have been added to substrates using endo-β-N-acetylglucosamines such as endo-F, endo-M (Wang et al., Tetrahedron Lett. 37: 1975-1978); and Haneda et al., Carbohydr. Res. 292: 61-70 (1996)).

In addition to their use in the preparing carbohydrates, the enzymes discussed above have been applied to the synthesis of glycoproteins as well. The synthesis of a homogenous glycoform of ribonuclease B has been published (Witte K. et al., J. Am. Chem. Soc. 119: 2114-2118 (1997)). The high mannose core of ribonuclease B was cleaved by treating the glycoprotein with endoglycosidase H. The cleavage occurred specifically between the two core GlcNAc residues. The tetrasaccharide sialyl Lewis X was then enzymatically rebuilt on the remaining GlcNAc anchor site on the now homogenous protein by the sequential use of β-1,4-galactosyltransferase, α-2,3-sialyltransferase and α-1,3-fucosyltransferase V. Each enzymatically catalyzed step proceeded in excellent yield.

Methods combining both chemical and enzymatic synthetic elements are also known. For example, Yamamoto and coworkers (Carbohydr. Res. 305: 415-422 (1998)) reported the chemoenzymatic synthesis of the glycopeptide, glycosylated Peptide T, using an endoglyosidase. The N-acetylglucosaminyl peptide was synthesized by purely chemical means. The peptide was subsequently enzymatically elaborated with the oligosaccharide of human transferrin glycopeptide. The saccharide portion was added to the peptide by treating it with an endo-β-N-acetylglucosaminidase. The resulting glycosylated peptide was highly stable and resistant to proteolysis when compared to the peptide T and N-acetylglucosaminyl peptide T.

In conjunction with the interest in the use of enzymes to form and remodel glycoproteins, there is interest in producing enzymes that are engineered to produce desired glycosylation patterns. Methods of producing and characterizing mutations of enzymes of use in producing glycoproteins have been reported. For example, Rao et al. (Protein Science 8:2338-2346 (1999)) have prepared mutants of endo-β-N-acetylglucosaminidase that are defined by structural changes, which reduce substrate binding and alter the enzyme functionality. Withers et al. (U.S. Pat. No. 5,716,812) have prepared mutant glycosidase enzymes in which the normal nucleophilic amino acid within the active site has been changed to a non-nucleophilic amino acid. The mutated enzymes cannot hydrolyze disaccharide products, but can still form them.

The overall structure and the structure of the active site of both mutated and native enzymes have been characterized by x-ray crystallography. See, e.g., van Roey et al., Biochemistry 33: 13989-13996 (1994); and Norris et al., Structure 2: 1049-1059 (1994).

C. Fucosylation

Many glycopeptides require the presence of particular fucosylated structures in order to exhibit biological activity. Intercellular recognition mechanisms often require a fucosylated oligosaccharide. For example, a number of proteins that function as cell adhesion molecules, including P-selectin, L-selectin, and E-selectin, bind specific cell surface fucosylated carbohydrate structures, for example, the sialyl Lewis x and the sialyl Lewis a structures. In addition, the specific carbohydrate structures that form the ABO blood group system are fucosylated. The carbohydrate structures in each of the three groups share a Fucα1,2Galβ1—dissacharide unit. In blood group O structures, this disaccharide is the terminal structure. The group A structure is formed by an α1,3 GalNAc transferase that adds a terminal GalNAc residue to the dissacharide. The group B structure is formed by an α1,3 galactosyltransferase that adds terminal galactose residue.

The Lewis blood group structures are also fucosylated. For example the Lewis x and Lewis a structures are Galβ1,4(Fucα1,3)GlcNac and Galβ1,4(Fucα1,4)GlcNac, respectively. Both these structures can be further sialylated (NeuAcα2,3-) to form the corresponding sialylated structures. Other Lewis blood group structures of interest are the Lewis y and b structures which are Fucα1,2Galβ1,4(Fucα1,3)GlcNAcβ-OR and Fucα1,2Galβ1,3(Fucα1,4)GlcNAc-OR, respectively. For a description of the structures of the ABO and Lewis blood group structures and the enzymes involved in their synthesis see, Essentials of Glycobiology, Varki et al. eds., Chapter 16 (Cold Spring Harbor Press, Cold Spring Harbor, N.Y., 1999).

Fucosyltransferases have been used in synthetic pathways to transfer a fucose unit from guanosine-5′-diphosphofucose to a specific hydroxyl of a saccharide acceptor. For example, Ichikawa prepared sialyl Lewis-X by a method that involves the fucosylation of sialylated lactosamine with a cloned fucosyltransferase (Ichikawa et al., J. Am. Chem. Soc. 114: 9283-9298 (1992)). Lowe has described a method for expressing non-native fucosylation activity in cells, thereby producing fucosylated glycoproteins, cell surfaces, etc. (U.S. Pat. No. 5,955,347).

Despite the many advantages of the enzymatic synthesis methods set forth above, in some cases, deficiencies remain. Since the biological activity of many commercially important recombinantly and transgenically produced glycopeptides depends upon the presence of a particular glycoform, or the absence of a particular glycoform, a need exists for an in vitro procedure to enzymatically modify glycosylation patterns, particularly fucosylation pattern, on such glycopeptides. The present invention fulfills these and other needs.

### SUMMARY OF THE INVENTION

The present invention provides methods for modifying the fucosylation pattern of glycopeptides. The methods include providing a glycopeptide that has an acceptor moiety for a fucosyltransferase and contacting the glycopeptide with a reaction mixture that comprises a fucose donor moiety and the fucosyltransferase under appropriate conditions to transfer fucose from the fucose donor moiety to the acceptor moiety, such that the glycopeptide has a substantially uniform fucosylation pattern.

Typically, in the method of the invention, at least about 60% of the targeted acceptor moieties are flicosylated and often at least about 80% of the targeted acceptor moieties on the glycopeptide are fucosylated. In some embodiments, the glycopeptide is reversibly immobilized on a solid support, such as an affinity chromatography medium.

The present invention also provides methods for producing glycopeptides that have a fucosylation pattern, which is substantially identical to the fucosylation pattern of a known glycopeptide. The method includes contacting a glycopeptide having an acceptor for a fucosyltransferase with a fucose donor and the fucosyltransferase. The transfer of the fucose onto the glycopeptide is terminated upon reaching a desired level of fucosylation. Among the uses of this aspect of the invention is the duplication of therapeutically relevant glycopeptide structures that have been approved or are nearing approval by a regulatory agency for use in humans. Thus, although a more thoroughly fucosylated peptide might have improved properties, the ability to duplicate an already approved glycopeptide structure obviates the necessity of submitting certain glycopeptides prepared by the instant method to the full regulatory review process, thereby providing an important economic advantage. This would allow switching from a production cell line with adequate glycosylation capabilities, but limited in expression level, to a production cell line that has the capability of producing significantly greater amounts of product, but yielding an inferior glycosylation pattern. The glycosylation pattern can then be modified in vitro to match that of the desired product. The yield of desired glycosylated product may then be increased substantially for a given bioreactor size, impacting both production economics and plant capacity. The particular glycopeptide used in the methods of the invention is generally not a critical aspect of the invention. The glycopeptide may be a fragment or a full-length glycopeptide. Typically, the glycopeptide is one that has therapeutic use such as a hormone, a growth factor, an enzyme inhibitor, a cytokine, a receptor, a IgG chimera, or a monoclonal antibody.

The fucosyltransferase may be eukaryotic or prokaryotic, and is usually mammalian or bacterial. In some embodiments, the preferred enzyme is bacterial. In other embodiments, a preferred fucosyltransferase is a FucT-VI, usually a mammalian FucT-VI. Alternatively, the fucosyltransferase is a FucT-VII, usually a mammalian FucT-VII. The fucosyltransferase may be isolated from its natural source organism or may be recombinantly produced. If recombinantly produced it may lack a membrane anchoring domain.

A number of acceptor moieties can be used, depending upon the particular enzyme used. Exemplary acceptor moieties include Galβ1-OR, Galβ1,3/4GlcNAc-OR, NeuAcα2,3Galβ1,3/4GlcNAc-OR, wherein R is an amino acid, a saccharide, an oligosaccharide or an aglycon group having at least one carbon atom and is linked to or is part of a glycopeptide.

Also provided are methods for the large-scale production of fucosylated glycopeptides having a substantially uniform fucosylation pattern, and large-scale methods for producing fucosylated glycopeptides having a known fucosylation pattern.

The invention also provides compositions comprising the glycopeptides fucosylated by the methods of the invention, and methods of using the composition in therapy and diagnosis.

Additional objects and advantages of the present invention will be apparent from the detailed description that follows.

### BRIEF DESCRIPTION OF THE DRAWINGS

FIG. 1 displays structures of exemplary O-linked selectin ligands.

FIG. 2 displays structures of exemplary N-linked selectin ligands.

FIG. 3 is the profile of produced by FACE analysis of N-glycans released from a glycopeptide prepared by a method of the invention.

FIG. 4 is the FACE analysis of a sialylation reaction performed prior to fucosylation by the method of the invention.

FIG. 5 is the FACE analysis of a glycopeptide fucosylated according to a method of the invention.

### DETAILED DESCRIPTION OF THE INVENTION AND THE PREFERRED EMBODIMENTS

Abbreviations

Ara, arabinosyl; Fru, fructosyl; Fuc, fucosyl; Gal, galactosyl; GalNAc, N-acetylgalacto; Glc, glucosyl; GlcNAc, N-acetylgluco; Man, mannosyl; ManAc, mannosyl acetate; Xyl, xylose; and NeuAc, sialyl (N-acetylneuraminyl); FucT, fucosyltransferase

Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

“Peptide” refers to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, β-alanine, phenylglycine and homoarginine are also included. Amino acids that are not gene-encoded may also be used in the present invention. Furthermore, amino acids that have been modified to include reactive groups may also be used in the invention. All of the amino acids used in the present invention may be either the D - or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

An “acceptor moiety” for a glycosyltransferase is an oligosaccharide structure that can act as an acceptor for a particular glycosyltransferase. When the acceptor moiety is contacted with the corresponding glycosyltransferase and sugar donor moiety, and other necessary reaction mixture components, and the reaction mixture is incubated for a sufficient period of time, the glycosyltransferase transfers sugar residues from the sugar donor moiety to the acceptor moiety. The acceptor moiety will often vary for different types of a particular glycosyltransferase. For example, the acceptor moiety for a mammalian galactoside 2-L-fucosyltransferase (α1,2-fucosyltransferase) will include a Galβ1,4-GlcNAc-R at a non-reducing terminus of an oligosaccharide; this fucosyltransferase attaches a fucose residue to the Gal via an α1,2 linkage. Terminal Galβ1,4-GlcNAc-R and Galβ1,3-GlcNAc-R and sialylated analogs thereof are acceptor moieties for α1,3 and α1,4-fucosyltransferases, respectively. These enzymes, however, attach the fucose to the GlcNAc residue of the acceptor. Accordingly, the term “acceptor moiety” is taken in context with the particular glycosyltransferase of interest for a particular application. Acceptor moieties for additional fucosyltransferases, and for other glycosyltransferases, are described herein.

A “substantially uniform glycoform” or a “substantially uniform glycosylation pattern,” when referring to a glycopeptide species, refers to the percentage of acceptor moieties that are glycosylated by the glycosyltransferase of interest (e.g., fucosyltransferase). For example, in the case of the α1,2 fucosyltransferase noted above, a substantially uniform facosylation pattern exists if substantially all (as defined below) of the Galβ1,4-GlcNAc-R and sialylated analogues thereof are moieties that are fucosylated in a composition comprising the glycopeptide of interest is calculated. It will be understood by one of skill in the art, that the starting material may contain glycosylated acceptor moieties (e.g., fucosylated Galβ1,4-GlcNAc-R moieties). Thus, the calculated percent glycosylation will include acceptor moieties that are glycosylated by the methods of the invention, as well as those acceptor moieties already glycosylated in the starting material.

The term “substantially” in the above definitions of “substantially uniform” generally means at least about 60%, at least about 70%, at least about 80%, or more preferably at least about 90%, and still more preferably at least about 95% of the acceptor moieties for a particular glycosyltransferase are glycosylated.

The term “substantially identical fucosylation pattern,” refers to a glycosylation pattern of a glycopeptide produced by a method of the invention which is at least about 80%, more preferably at least about 90%, even more preferably at least about 95% and still more preferably at least about 98% identical to the fucosylation of a known glycoprotein. “Known fucosylation pattern,” refers to a fucosylation pattern of a known glycopeptide from any source having any known level of fucosylation.

The term “sialic acid” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano etal. (1986)J. Biol. Chem. 261: 11550-11557; Kanamori et al., J. Biol. Chem. 265: 21811-21819 (1990)). Also included are 9-substituted sialic acids such as a 9-O-C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids: Chemistry, Metabolism and Function, R. Schauer, Ed. (Springer-Verlag, New York (1992)). The synthesis and use of sialic acid compounds in a sialylation procedure is disclosed in international application WO 92/16640, published Oct. 1, 1992.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques. A “recombinant polypeptide” is one which has been produced by a recombinant cell.

A “heterologous sequence” or a “heterologous nucleic acid”, as used herein, is one that originates from a source foreign to the particular host cell, or, if from the same source, is modified from its original form. Thus, a heterologous glycopeptide gene in a eukaryotic host cell includes a glycopeptide-encoding gene that is endogenous to the particular host cell that has been modified. Modification of the heterologous sequence may occur, e.g., by treating the DNA with a restriction enzyme to generate a DNA fragment that is capable of being operably linked to the promoter. Techniques such as site-directed mutagenesis are also useful for modifying a heterologous sequence.

A “subsequence” refers to a sequence of nucleic acids or amino acids that comprise a part of a longer sequence of nucleic acids or amino acids (e.g., polypeptide) respectively.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The term “isolated” refers to material that is substantially or essentially free from components which interfere with the activity of an enzyme. For cells, saccharides, nucleic acids, and polypeptides of the invention, the term “isolated” refers to material that is substantially or essentially free from components which normally accompany the material as found in its native state. Typically, isolated saccharides, proteins or nucleic acids of the invention are at least about 80% pure, usually at least about 90%, and preferably at least about 95% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized.

The practice of this invention can involve the construction of recombinant nucleic acids and the expression of genes in transfected host cells. Molecular cloning techniques to achieve these ends are known in the art. A wide variety of cloning and in vitro amplification methods suitable for the construction of recombinant nucleic acids such as expression vectors are well known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1999 Supplement) (Ausubel). Suitable host cells for expression of the recombinant polypeptides are known to those of skill in the art, and include, for example, eukaryotic cells including insect, mammalian and fungal cells.

Examples of protocols sufficient to direct persons of skill through in vitro amplification methods, including the polymerase chain reaction (PCR) the ligase chain reaction (LCR), Qβ-replicase amplification and other RNA polymerase mediated techniques are found in Berger, Sambrook, and Ausubel, as well as Mullis et al. (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039.

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right.

All oligosaccharides described herein are described with the name or abbreviation for the non-reducing saccharide (i.e., Gal), followed by the configuration of the glycosidic bond (α or .β), the ring bond (1 or 2), the ring position of the reducing saccharide involved in the bond (2, 3, 4, 6 or 8), and then the name or abbreviation of the reducing saccharide (i.e., GlcNAc). Each saccharide is preferably a pyranose. For a review of standard glycobiology nomenclature see, Essentials of Glycobiology Varki et al. eds. CSHL Press (1999)

Introduction

Glycopeptides that have modified glycosylation patterns generally have important advantages over peptides that are in their unaltered glycosylation state, or that are in a glycosylation state that is less than optimal for a particular application. Such non-optimal glycosylation patterns can arise, for example, when a recombinant glycopeptide is produced in a cell that does not have the proper complement of glycosylation machinery to produce the desired glycosylation pattern. The optimal or preferred glycosylation pattern may or may not be the native glycosylation pattern of the glycopeptide when produced in its native cell.

The biological activity of some glycopeptides depends upon the presence or absence of a particular glycoform. For example, increased glycosylation at an acceptor moiety will render a glycopeptide highly multivalent, thereby increasing the biological activity of the altered glycopeptide. Other advantages of glycopeptide compositions that have altered glycosylation patterns include, for example, increased therapeutic half-life of a glycopeptide due to reduced clearance rate. Altering the glycosylation pattern can also mask antigenic determinants on foreign proteins, thus reducing or eliminating an immune response against the protein. Alteration of the glycoform of a glycopeptide-linked saccharide can also be used to target a protein to a particular tissue or cell surface receptor that is specific for the altered oligosaccharide. The altered oligosaccharide can also be used as an inhibitor of the receptor with its natural ligand. The present invention provides enzymatic methods for modifying the fucosylation pattern of glycopeptides.

The Methods

The present invention provides methods of producing glycopeptide species having a selected glycosylation pattern.

In a first aspect, the invention provides a method for producing a population of glycopeptides in which the members of the population have a substantially uniform glycosylation pattern. In particular, the invention provides methods for preparing glycopeptides that have a substantially uniform fucosylation pattern. In some embodiments, other glycosyltransferases can be used in combination with fucosyltransferases to produce desired glycosylation patterns. Methods and kits for practicing the methods of the invention are also provided. The methods of the invention are useful for altering the glycosylation pattern of a glycopeptide from that which is present on the glycopeptide upon its initial expression. In a particularly preferred embodiment, the fucosylation pattern of a collection of copies of a glycoprotein is homogeneous; each copy has substantially the same fucosylation pattern.

The methods provided by the invention for attaching saccharide residues to glycopeptides can, unlike previously described glycosylation methods, provide a population of a glycopeptide in which the members have a substantially uniform glycosylation pattern. Thus, in preferred embodiments, the population of glycopeptides is substantially monodisperse vis-a-vis the fucosylation pattern of each member of the population. After application of the methods of the invention, a desired saccharide residue (e.g., a fucosyl residue) will be attached to a high percentage of acceptor moieties.

The invention also provides a method for reproducing a known glycosylation pattern on a peptide substrate. The method includes glycosylating the substrate to a preselected (i.e., known) level, at which point the glycosylation is stopped. In a particularly preferred embodiment, the peptide substrate is fucosylated to a known level. The method of the invention is of particular use in preparing glycoproteins that are replicas of therapeutic proteins, which are presently used clinically or are advanced in clinical trials.

Both of the above described methods are also practical for large-scale production of modified glycopeptides, including both pilot scale and industrial scale preparations. Thus, the methods of the invention provide a practical means for large-scale preparation of glycopeptides having altered fucosylation patterns. The methods are well suited for modification of therapeutic glycopeptides that are incompletely, or improperly, glycosylated during production in cells (e.g., mammalian cells or transgenic animals). The processes provide an increased and consistent level of a desired glycoform on glycopeptides present in a composition.

a. The Substrates

The methods of the invention can be practiced using any fucosylation substrate that includes an acceptor moiety for a fucosyltransferase. Exemplary substrates include, but are not limited to, peptides, gangliosides and other biological structures (e.g., glycolipids, whole cells, and the like) that can be modified by the methods of the invention. Exemplary structures, which can be modified by the methods of the invention include any a of a number glycopeptides and carbohydrate structures on cells known to those skilled in the art as set forth is Table 1.

TABLE 1

| | |
|---|---|
| Hormones and Growth | Receptors and Chimeric Receptors |
| Factors | CD4 |
| G-CSF | Tumor Necrosis Factor (TNF) receptor |
| GM-CSF | Alpha-CD20 |
| TPO | MAb-CD20 |
| EPO | MAb-alpha-CD3 |
| EPO variants | MAb-TNF receptor |
| alpha-TNF | MAb-CD4 |
| Leptin | PSGL-1 |
| Enzymes and Inhibitors | MAb-PSGL-1 |
| t-PA | Complement |
| t-PA variants | GlyCAM or its chimera |
| Urokinase | N-CAM or its chimera |
| Factors VII, VIII, IX, X | LFA-3 |
| DNase | CTLA-IV |
| Glucocerebrosidase | Monoclonal Antibodies (Immunoglobulins) |
| Hirudin | MAb-anti-RSV |
| α1 antitrypsin | MAb-anti-IL-2 receptor |
| Antithrombin III | MAb-anti-CEA |
| Cytokines and Chimeric | MAb-anti-platelet IIb/IIIa receptor |
| Cytokines | MAb-anti-EGF |
| Interleukin-1 (IL-1), 1B, | MAb-anti-Her-2 receptor |
| 2, 3, 4 | Cells |
| Interferon-alpha (IFN- | Red blood cells |
| alpha) | White blood cells (e.g., T cells, B cells, |
| IFN-alpha-2b | dendritic cells, |
| IFN-beta | macrophages, NK cells, neutrophils, monocytes |
| IFN-gamma | and the like |
| Chimeric diptheria | Stem cells |
| toxin-IL-2 | |

Peptides that are modified by methods of the invention include, but are not limited to, members of the immunoglobulin family (e.g., antibodies, MHC molecules, T cell receptors, and the like), intercellular receptors (e.g., integrins, receptors for hormones or growth factors and the like) lectins, and cytokines (e.g., interleukins). Other examples include tissue-type plasminogen activator (t-PA), renin, clotting factors such as factor VIII and factor IX, bombesin, thrombin, hematopoietic growth factor, colony stimulating factors, viral antigens, glycosyltransferases, and the like. Polypeptides of interest for recombinant expression and subsequent modification using the methods of the invention also include complement proteins, α1-antitrypsin, erythropoietin, P-selectin glycopeptide ligand-1 (PSGL-1), granulocyte-macrophage colony stimulating factor, anti-thrombin III, interleukins, interferons, proteins A and C, fibrinogen, herceptin, leptin, glycosidases, among many others. This list of polypeptides is exemplary, not exclusive.

The methods are also useful for modifying the glycosylation patterns of chimeric proteins, including, but not limited to, chimeric proteins that include a moiety derived from an immunoglobulin, such as IgG. Methods of preparing IgG chimeras are known in the art (see, for example, ANTIBODY FUSION PROTEINS; Edited by Steven M. Chamow and Avi Ashkenazi).

Altering the glycosylation pattern of immunoglobulins, as well as chimeric peptides that include all or part of an immunoglobulin, such as an immunoglobulin heavy chain constant region, also provides enhanced biological activity. Oligosaccharides attached to IgG molecules purified from human sera, in particular the oligosaccharides attached to Asn297 of IgG, are important for IgG structure and function (Rademacher et al., Prog. Immunol 5: 95-112 (1983)). The absence of these oligosaccharides results in a lack of binding to the monocyte Fc receptor, a decline in complement activation, an increase in susceptibility to proteolytic degradation, and reduced clearance from circulation of antibody-antigen complexes. Immunoglobulin oligosaccharides, in particular those of IgG, naturally exhibit high microheterogeneity in their structures (Kobata, Glycobiology 1: 5-8 (1990)). Therefore, use of the methods of the invention to provide a more uniform glycopeptide results in an improvement of one or more of these biological activities (e.g., enhanced complement activation, increased binding to the monocyte Fc receptor, reduced proteolysis, and increased clearance of antibody-antigen complexes). The methods of the invention are also useful for modifying oligosaccharides on other immunoglobulins to enhance one or more biological activities. For example, high-mannose oligosaccharides are generally attached to IgM and IgD. Such oligosaccharides can be modified as described herein to yield antibodies with enhanced properties.

b. Glycosyltransferases and Methods for Preparing Compositions of Glycopeptides Having Selected Glycosylation Patterns

The methods of the invention utilize glycosyltransferases (e.g., fucosyltransferases) that are selected for their ability to produce glycopeptides having a selected glycosylation pattern. For example, glycosyltransferases are selected that not only have the desired specificity, but also are capable of glycosylating a high percentage of desired acceptor groups in a glycopeptide preparation. It is preferable to select a glycosyltransferase based upon results obtained using an assay system that employs an oligosaccharide acceptor moiety that is attached to a glycopeptide, in contrast to a soluble oligosaccharide or an oligosaccharide that is attached to a relatively short peptide. The use of glycosylation assay results on a glycopeptide-linked oligosaccharide is advantageous because results obtained using short peptides or soluble oligosaccharides are often not predictive of the activity of a glycosyltransferase on a glycopeptide-linked oligosaccharide. One can use the particular glycopeptide of interest in the assay to identify a suitable glycosyltransferase. One can, however, also use a “standard” glycopeptide, i.e., a readily available glycopeptide that has a linked oligosaccharide, which includes an acceptor moiety for the glycosyltransferase of interest.

In certain embodiments, the glycosyltransferase is a fusion protein. Exemplary fusion proteins include glycosyltransferases that exhibit the activity of two different glycosyltransferases (e.g., sialyltransferase and fucosyltransferase). Other fusion proteins will include two different variations of the same transferase activity (e.g., FucT-VI and FucT-VII). Still other fusion proteins will include a domain that enhances the utility of the transferase activity (e.g, enhanced solubility, stability, turnover, enhanced expression, affinity tag for removal of transferase, etc.).

Examples of suitable glycosyltransferases for use in the preparation of the compositions of the invention are described herein. One can readily identify other suitable glycosyltransferases by reacting various amounts of each enzyme (e.g., 1-100 mU/mg protein) with a glycopeptide (e.g., at 1-10 mg/ml) to which is linked an oligosaccharide that has a potential acceptor site for the glycosyltransferase of interest. The abilities of the glycosyltransferases to add a sugar residue at the desired site are compared, and a glycosyltransferase having the desired property is selected for use in a method of the invention.

In some embodiments, it is advantageous to use a glycosyltransferase that provides the desired glycoform using a low ratio of enzyme units to glycopeptide. In some embodiments, the desired glycosylation will be obtained using about 50 mU or less of glycosyltransferase per mg of glycopeptide. Preferably, less than about 40 mU of glycosyltransferase is used per mg of glycopeptide, even more preferably, the ratio of glycosyltransferase to glycopeptide is less than or equal to about 35 mU/mg, and more preferably it is about 25 mU/mg or less. Most preferably from an enzyme cost standpoint, the desired glycosylation will be obtained using less than about 10 mU/mg glycosyltransferase per mg glycopeptide. Typical reaction conditions will have glycosyltransferase present at a range of about 5-25 mU/mg of glycopeptide, or 10-50 mU/ml of reaction mixture with the glycopeptide present at a concentration of at least about 1-2 mg/ml. In a multi-enzyme reaction, these amounts of enzyme can be increased proportionally to the number of glycosyltransferases, sulfotransferases, or trans-sialidases.

In other embodiments, it is desirable to use a greater amount of enzyme. For example, to obtain a faster rate of reaction, one can increase the amount of enzyme by about 2-10-fold. The temperature of the reaction can also be increased to obtain a faster reaction rate. Generally, however, a temperature of about 30 to about 37° C., for example, is suitable.

The efficacy of the methods of the invention can be enhanced through use of recombinantly produced glycosyltransferases. Recombinant technique enable production of glycosyltransferases in the large amounts that are required for large-scale glycopeptide modification. Deletion of the membrane-anchoring domain of glycosyltransferases, which renders the glycosyltransferases soluble and thus facilitates production and purification of large amounts of glycosyltransferases, can be accomplished by recombinant expression of a modified gene encoding the glycosyltransferases. For a description of methods suitable for recombinant production of glycosyltransferases see, U.S. Pat. No. 5,032,519.

Also provided by the invention are glycosylation methods in which the target glycopeptide is immobilized on a solid support. The term “solid support” also encompasses semi-solid supports. Preferably, the target glycopeptide is reversibly immobilized so that the glycopeptide can be released after the glycosylation reaction is completed. Many suitable matrices are known to those of skill in the art. Ion exchange, for example, can be employed to temporarily immobilize a glycopeptide on an appropriate resin while the glycosylation reaction proceeds. A ligand that specifically binds to the glycopeptide of interest can also be used for affinity-based immobilization. Antibodies that bind to a glycopeptide of interest are suitable; where the glycopeptide of interest is itself an antibody or contains a fragment thereof, one can use protein A or G as the affinity resin. Dyes and other molecules that specifically bind to a protein of interest that is to be glycosylated are also suitable.

Proteins that are recombinantly produced are often expressed as a fusion protein that has a “tag” at one end, which facilitates purification of the glycopeptide. Such tags can also be used for immobilization of the protein while a glycosylation reaction is accomplished. Suitable tags include “epitope tags,” which are a polypeptide sequence that is specifically recognized by an antibody. Epitope tags are generally incorporated into fusion proteins to enable the use of a readily available antibody to unambiguously detect or isolate the fusion protein. A “FLAG tag” is a commonly used epitope tag, specifically recognized by a monoclonal anti-FLAG antibody, consisting of the sequence AspTyrLysAspAspAsp AspLys or a substantially identical variant thereof. Other suitable tags are known to those of skill in the art, and include, for example, an affinity tag such as a hexahistidine peptide, which will bind to metal ions such as nickel or cobalt ions.

Preferably, when the peptide portion of the glycopeptide is a truncated version of the full-length peptide, it preferably includes the biologically active portion of the full-length glycopeptide. Exemplary biologically active portions include, but are not limited to, enzyme active sites, receptor binding sites, ligand binding sites, complementarity determining regions of antibodies, and antigenic regions of antigens.

1. Fucosyltransferase Reactions

The invention provides methods of producing glycopeptides, which have a substantially uniform fucosylation pattern. For example, in some embodiments the glycoproteins produced by the methods of the have one or more oligosaccharide groups that are targeted acceptor moieties for a fucosyltransferase, in which at least 60%, preferably at least 80%, more preferably at least 90% and even more preferably at least 95% of the targeted acceptor moieties in the composition are fucosylated.

The methods of the invention are practiced by contacting a composition that includes multiple copies of a glycopeptide species, a majority of which preferably have one or more linked oligosaccharide groups that include an acceptor moiety for a fucosyltransferase, with a reaction mixture that includes a fucose donor moiety, a fucosyltransferase, and other reagents required for fucosyltransferase activity. The glycopeptide is incubated in the reaction mixture for a sufficient time and under appropriate conditions to transfer fucose from the fucose donor moiety to the fucosyltransferase acceptor moiety.

The fucosyltransferase used in the methods of the invention is chosen based upon its ability to fucosylate a selected percentage of the fucosyltransferase acceptor moieties of interest. Preferably, the fucosyltransferase is assayed for suitability in the methods of the invention using a fucosyltransferase acceptor moiety that is attached to a glycopeptide. The use of a glycopeptide-linked acceptor moiety, rather than an acceptor moiety that is part of a soluble oligosaccharide, in the assay to determine fucosyltransferase activity allows one to select a fucosyltransferase that produces the selected fucosylation pattern on the glycopeptide.

A number of fucosyltransferases are known to those of skill in the art. Briefly, fucosyltransferases include any of those enzymes, which transfer L-fucose from GDP-fucose to a hydroxy position of an acceptor sugar. In some embodiments, for example, the acceptor sugar is a GlcNAc in a Galβ(1→3,4)GlcNAc group in an oligosaccharide glycoside. Suitable fucosyltransferases for this reaction include the known Galβ(1→3,4)GlcNAc α(1→3,4)fucosyltransferase (FucT-III E.C. No. 2.4.1.65) which is obtained from human milk (see, e.g., Palcic et al., Carbohydrate Res. 190:1-11 (1989); Prieels, et al., J. Biol. Chem. 256:10456-10463 (1981); and Nunez, et al., Can. J. Chem. 59:2086-2095 (1981)) and the βGal(1→4)βGlcNAc α(1→3)fucosyltransferases (FucT-IV, FucT-V, FucT-VI, and FucT-VII, E.C. No. 2.4.1.65) which are found in human serum. A recombinant form of βGal(1→3,4)βGlcNAc α(1→3,4)fucosyltransferase is also available (see, Dumas, et al., Bioorg. Med. Letters 1: 425-428 (1991) and Kukowska-Latallo, et al., Genes and Development 4: 1288-1303 (1990)). Other exemplary fucosyltransferases include α1,2 fucosyltransferase (E.C. No. 2.4.1.69). Enzymatic fucosylation may be carried out by the methods described in Mollicone et al., Eur. J. Biochem. 191:169-176 (1990) or U.S. Pat. No. 5,374,655; an α1,3-fucosyltransferase from Schistosoma mansoni (Trottein et al. (2000) Mol. Biochem. Parasitol. 107: 279-287); and an α1,3 fucosyltransferase IX (nucleotide sequences of human and mouse FucT-IX are described in Kaneko et al. (1999) FEBS Lett. 452: 237-242, and the chromosomal location of the human gene is described in Kaneko et al. (1999) Cytogenet. Cell Genet. 86: 329-330. Recently reported α1,3-fucosyltransferases that use an N-linked GlcNAc as an acceptor from the snail Lymnaea stagnalis and from mung bean are described in van Tetering et al. (1999) FEBS Lett. 461: 311-314 and Leiter et al. (1999) J. Biol. Chem. 274: 21830-21839, respectively. In addition, bacterial fucosyltransferases such as the α(1,3/4) fucosyltransferase of Helicobacter pylori as described in Rasko et al. (2000) J. Biol. Chem. 275:4988-94, as well as the α1,2-fucosyltransferase of H. Pylori (Wang et al. (1999) Microbiology. 145: 3245-53. See, also Staudacher, E. (1996) Trends in Glycoscience and Glycotechnology, 8: 391-408, http://afmb.cnrs-mrs.fr/˜pedro/CAZY/gtf.html and http://www.vei.co.uk/TGN/gt—guide.htm for lists and descriptions of fucosyltransferases useful in the invention.

Exemplary fucosyltransferases of use in the present invention are provided in Table 2.

TABLE 2

| Fucosyl- | Other | Tissue | | |
|---|---|---|---|---|
| transferase | names | Distribution | Substrates | Products |
| FucT-III | Lewis a1-3/4 | milk, gall | type I, type II, | Le^{a}, SLe^{a,} |
| | Fuc-T | bladder, | sialyl type | Lex, |
| | | kidney, | I + II, fucosyl | SLe^{x},Le^{y}, |
| | | colon | type I + II, | SLe^{y}, |
| | | | lactose, 2- | VIM-2 |
| | | | fucosyl- | |
| | | | lactose | |
| FucT-IV | myeloid-type | brain, myeloid | type II, sialyl | Le^{x}, SLe^{x}, |
| | ELFT, ELAM- | cells | type II | VIM-2 |
| | I, ligand Fuc- | | | |
| | T | | | |
| FucT-V | plasma-type | plasma, milk, | type II, sialyl | Le^{x}, |
| | | liver | type II, type I, | SLe^{x}, |
| | | | lactose, 2- | SLe^{y}, |
| | | | fucosyl- | VIM-2 |
| | | | lactose | |
| FucT-VI | second | plasma, kidney, | type II, sialyl- | Le^{x}, SLe^{x}, |
| | plasma type | liver | type II, | ^{SLe}^{y} |
| | | | fucosyl-type | |
| | | | II | |
| FucT-VII | second | leukocytes | sialyl-type II | SLe^{x} |
| | myeloid | | | |

In some embodiments, the fucosyltransferase that is employed in the methods of the invention has an activity of at least about 1 Unit/ml, usually at least about 5 Units/ml.

In other embodiments, fucosyltransferases for use in the methods of the invention include FucT-VII and FucT-VI. Each of these enzymes preferably catalyzes the fucosylation of at least 60% of their targeted glycopeptide-linked fucosyltransferase acceptor sites present in a population of glycopeptides.

As most of the studies on in vitro fucosylation to date have focused on the fucosylation of small molecule substrates, the art has not recognized any substantial difference between the efficiency of fucosylation of the various fucosyltransferases. The inventors have, however, discovered that certain FucT molecules are surprisingly more effective at fucosylating glycopeptides. For example, FucT-VI is approximately 8-fold more effective at fucosylating glycopeptides than is FucT-V. Thus, in a preferred embodiment, the invention provides a method of fucosylating an acceptor on a glycopeptide using a fucosyltransferase that provides a degree of fucosylation that is at least about 2-fold greater, more preferably at least about 4-fold greater, still more preferably at least about 6-fold greater, and even more preferably at least about 8-fold greater than is achieved under identical conditions using FucT-V. Presently preferred fucosyltransferases include FucT-VI and FucT-VII.

Specificity for a selected substrate is only the first criterion a fucosyltransferase preferably satisfies. In a still further preferred embodiment, the fucosyltransferase is useful in a method for fucosylating a commercially important recombinant or transgenic glycopeptide. The fucosyltransferase used in the method of the invention is preferably also able to efficiently fucosylate a variety of glycopeptides, and support scale-up of the reaction to allow the fucosylation of at least about 500 mg of the glycoprotein. More preferably, the fucosyltransferase will support the scale of the fucosylation reaction to allow the synthesis of at least about 1 kg, and more preferably, at least 10 kg of recombinant glycopeptide with relatively low cost and infrastructure requirements.

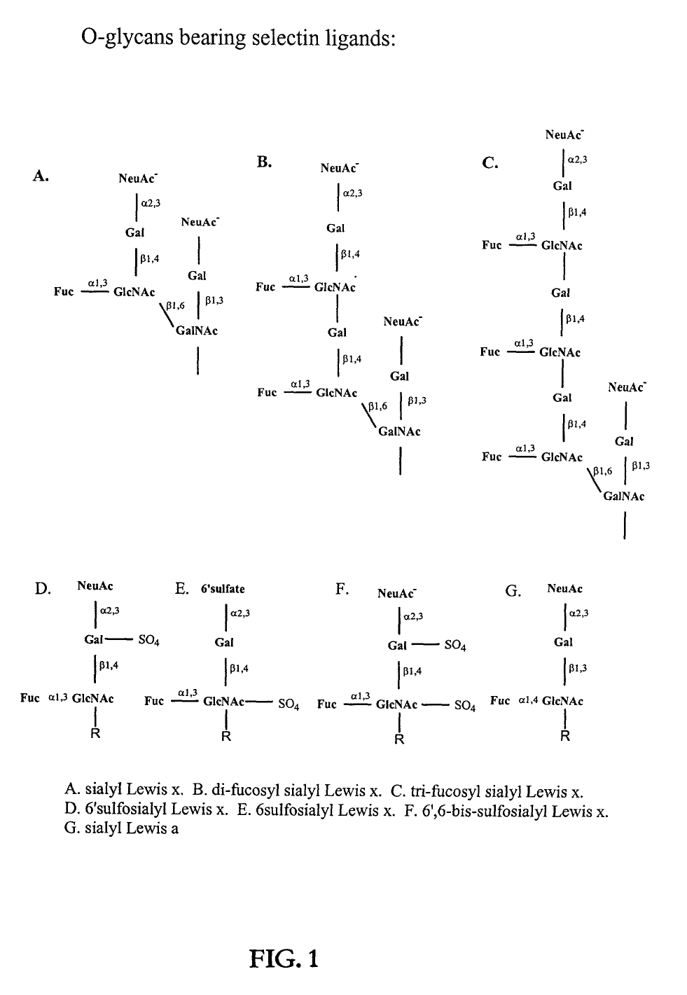

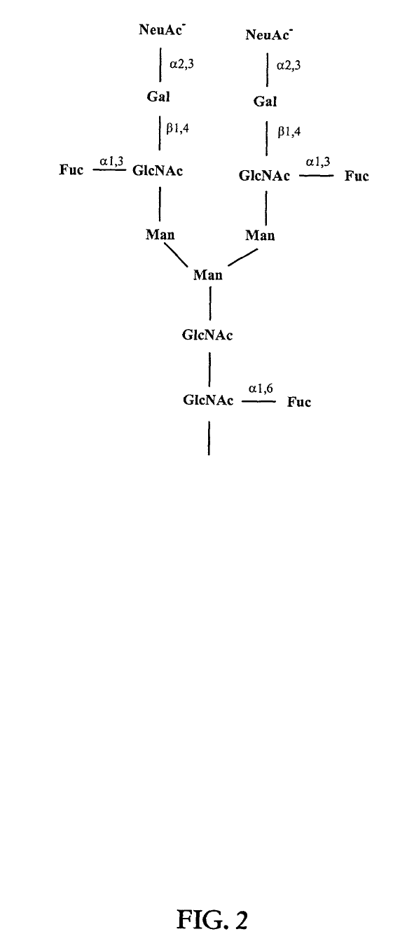

In an exemplary embodiment, the method of the invention results in the formation on a glycopeptide of at least one ligand for a selectin. Exemplary O-linked selectin ligands are set forth in FIG. 1. Exemplary N-linked selectin ligands are set forth in FIG. 2. Confirmation of the formation of the ligand is assayed in an operational manner by probing the ability of the glycopeptide to interact with a selectin. The interaction between a glycopeptide and a specific selectin is measureable by methods familiar to those in the art (see, for example, Jutila et al., J. Immunol. 153: 3917-28 (1994); Edwards et al., Cytometry 43(3): 211-6 (2001); Stahn et al., Glycobiology 8: 311-319 (1998); Luo et al., J. Cell Biochem. 80(4):522-31 (2001); Dong et al., J. Biomech. 33(1): 35-43 (2000); Jung et al., J. Immunol. 162(11): 6755-62 (1999); Keramidaris et al., J. Allergy Clin. Immunol. 107(4): 734-8 (2001); Fieger et al., Biochim. Biophys. Acta 1524(1): 75-85 (2001); Bruehl et al., J. Biol. Chem. 275(42): 32642-8 (2000); Tangemann et al., J. Exp. Med. 190(7): 935-42 (1999); Scalia et al., Circ. Res. 84(1): 93-102 (1999); Alon et al., J. Cell Biol. 138(5): 1169-80 (1997); Steegmaier et al., Eur. J. Immunol. 27(6): 1339-45 (1997); Stewart et al., J. Med. Chem. 44(6): 988-1002 (2001); Schurmann et al, Gut 36(3): 411-8 (1995); Burrows et al., J. Clin. Pathol. 47(10): 939-44 (1994)).

Suitable acceptor moieties for fucosyltransferase-catalyzed attachment of a fucose residue include, but are not limited to, GlcNAc-OR, Galβ1,3GlcNAc-OR, NeuAcα2,3Galβ1,3GlcNAc-OR, Galβ1,4GlcNAc-OR and NeuAcα2,3Galβ1,4GlcNAc-OR, where R is an amino acid, a saccharide, an oligosaccharide or an aglycon group having at least one carbon atom. R is linked to or is part of a glycopeptide. The appropriate fucosyltransferase for a particular reaction is chosen based on the type of fucose linkage that is desired (e.g., α2, α3, or α4), the particular acceptor of interest, and the ability of the facosyltransferase to achieve the desired high yield of fucosylation. Suitable ficosyltransferases and their properties are described above.

If a sufficient proportion of the glycopeptide-linked oligosaccharides in a composition does not include a fucosyltransferase acceptor moiety, one can synthesize a suitable acceptor. For example, one preferred method for synthesizing an acceptor for a fucosyltransferase involves use of a GlcNAc transferase to attach a GlcNAc residue to a GlcNAc transferase acceptor moiety, which is present on the glycopeptide-linked oligosaccharides. In preferred embodiments a transferase is chosen, having the ability to glycosylate a large fraction of the potential acceptor moieties of interest. The resulting GlcNAcβ-OR can then be used as an acceptor for a fucosyltransferase.

The resulting GlcNAcβ-OR moiety can be galactosylated prior to the fucosyltransferase reaction, yielding, for example, a Galβ1,3GlcNAc-OR or Gal β1,4GlcNAc-OR residue. In some embodiments, the galactosylation and fucosylation steps are carried out simultaneously. By choosing a fucosyltransferase that requires the galactosylated acceptor, only the desired product is formed. Thus, this method involves:

(a) galactosylating a compound of the formula GlcNAcβ-OR with a galactosyltransferase in the presence of a UDP-galactose under conditions sufficient to form the compounds Galβ1,4GlcNAcβ-OR or Galβ1,3GlcNAc-OR; and

(b) fucosylating the compound formed in (a) using a fucosyltransferase in the presence of GDP-fucose under conditions sufficient to form a compound selected from:

Fucα1,2Galβ1,4GlcNAc1β-O1R;

Fucα1,2Galβ1,3GlcNAc-OR;

Galβ1,4(Fuc1,α3)GlcNAcβ-OR; or

Galβ1,3(Fucα1,4)GlcNAc-OR.

One can add additional fucose residues to a fucosylated glycopeptide treating the fucosylated peptide with a fucosyltransferase, which has the desired activity. For example, the methods can form oligosaccharide determinants such as Fucα1,2Galβ1,4(Fucα1,3)GlcNAcβ-OR and Fucα1,2Galβ1,3(Fucα1,4)GlcNAc-OR. Thus, in another preferred embodiment, the method includes the use of at least two fucosyltransferases. The multiple fucosyltransferases are used either simultaneously or sequentially. When the fucosyltransferases are used sequentially, it is generally preferred that the glycoprotein is not purified between the multiple facosylation steps. When the multiple fucosyltransferases are used simultaneously, the enzymatic activity can be derived from two separate enzymes or, alternatively, from a single enzyme having more than one fucosyltransferase activity.

2. Multiple-enzyme Oligosaccharide Synthesis

As discussed above, in some embodiments, two or more enzymes are used to form a desired oligosaccharide determinant. For example, a particular oligosaccharide determinant might require addition of a galactose, a sialic acid, and a fucose in order to exhibit a desired activity. Accordingly, the invention provides methods in which two or more enzymes, e.g., glycosyltransferases, trans-sialidases, or sulfotransferases, are used to obtain high-yield synthesis of a desired oligosaccharide determinant.

In a particularly preferred embodiment, one of the enzymes used is a sulfotransferase which sulfonates the saccharide or the peptide. Even more preferred is the use of a sulfotransferase to prepare a ligand for a selectin (Kimura et al., Proc Natl Acad Sci U S A 96(8):4530-5 (1999)).

In some cases, a glycopeptide-linked oligosaccharide will include an acceptor moiety for the particular glycosyltransferase of interest upon in vivo biosynthesis of the glycopeptide. Such glycopeptides can be glycosylated using the methods of the invention without prior modification of the glycosylation pattern of the glycopeptide. In other cases, however, a glycopeptide of interest will lack a suitable acceptor moiety. In such cases, the methods of the invention can be used to alter the glycosylation pattern of the glycopeptide so that the glycopeptide-linked oligosaccharides then include an acceptor moiety for the glycosyltransferase-catalyzed attachment of a preselected saccharide unit of interest to form a desired oligosaccharide determinant.

Glycopeptide-linked oligosaccharides optionally can be first “trimmed,” either in whole or in part, to expose either an acceptor moiety for the glycosyltransferase or a moiety to which one or more appropriate residues can be added to obtain a suitable acceptor. Enzymes such as glycosyltransferases and endoglycosidases are useful for the attaching and trimming reactions. For example, a glycopeptide that displays “high mannose”-type oligosaccharides can be subjected to trimming by a mannosidase to obtain an acceptor moiety that, upon attachment of one or more preselected saccharide units, forms the desired oligosaccharide determinant.

The methods are also useful for synthesizing a desired oligosaccharide moiety on a protein that is unglycosylated in its native form. A suitable acceptor for the corresponding glycosyltransferase can be attached to such proteins prior to glycosylation using the methods of the present invention. See, e.g., U.S. Pat. No. 5,272,066 for methods of obtaining polypeptides having suitable acceptors for glycosylation.

Thus, in some embodiments, the invention provides methods for in vitro sialylation of saccharide groups present on a glycopeptide that first involves modifying the glycopeptide to create a suitable acceptor. Examples of preferred methods of multi-enzyme synthesis of desired oligosaccharide determinants are as follows.

(i). Fucosylated and sialylated oligosaccharide determinants

Oligosaccharide determinants that confer a desired biological activity upon a glycopeptide often are sialylated in addition to being fucosylated. Accordingly, the invention provides methods in which a glycopeptide-linked oligosaccharide is sialylated and fucosylated in high yields.

The sialylation can be accomplished using either a trans-sialidase or a sialyltransferase, except where a particular determinant requires an α2,6-linked sialic acid, in which a sialyltransferase is used. Suitable examples of each type of enzyme are described above. These methods involve sialylating an acceptor for a sialyltransferase or a trans-sialidase by contacting the acceptor with the appropriate enzyme in the presence of an appropriate donor moiety. For sialyltransferases, CMP-sialic acid is a preferred donor moiety. Trans-sialidases, however, preferably use a donor moiety that includes a leaving group to which the trans-sialidase cannot add sialic acid.

Acceptor moieties of interest include, for example, Galβ-OR. In some embodiments, the acceptor moieties are contacted with a sialyltransferase in the presence of CMP-sialic acid under conditions in which sialic acid is transferred to the non-reducing end of the acceptor moiety to form the compound NeuAcα2,3Galβ-OR or NeuAcα2,6Galβ-OR. In this formula, R is an amino acid, a saccharide, an oligosaccharide or an aglycon group having at least one carbon atom. R is linked to or is part of a glycopeptide. An β2,8-sialyltransferase can also be used to attach a second or multiple sialic acid residues to the above structures.

To obtain an oligosaccharide determinant that is both sialylated and fucosylated, the sialylated acceptor is contacted with a fucosyltransferase as discussed above. The sialyltransferase and fucosyltransferase reactions are generally conducted sequentially, since most sialyltransferases are not active on a fucosylated acceptor. FucT-VII, however, acts only on a sialylated acceptor. Therefore, FucT-VII can be used in a simultaneous reaction with a sialyltransferase.

If the trans-sialidase is used to accomplish the sialylation, the fucosylation and sialylation reactions can be conducted either simultaneously or sequentially, in either order. The peptide to be modified is incubated with a reaction mixture that contains a suitable amount of a trans-sialidase, a suitable sialic acid donor substrate, a fucosyltransferase (capable of making an α1,3 or α1,4 linkage), and a suitable fucosyl donor substrate (e.g., GDP-fucose).

(ii). Galactosylated, fucosylated and sialylated oligosaccharide determinants

The invention also provides methods for synthesizing oligosaccharide determinants that are galactosylated, fucosylated, and sialylated. Either a sialyltransferase or a trans-sialidase (for α2,3-linked sialic acid only) can be used in these methods.

The trans-sialidase reaction involves incubating the protein to be modified with a reaction mixture that contains a suitable amount of a galactosyltransferase (galβ1,3 or galβ1,4), a suitable galactosyl donor (e.g., UDP-galactose), a trans-sialidase, a suitable sialic acid donor substrate, a fucosyltransferase (capable of making an α1,3 or α1,4 linkage), a suitable facosyl donor substrate (e.g., GDP-fucose), and a divalent metal ion. These reactions can be carried out either sequentially or simultaneously.

If a sialyltransferase is used, the method involves incubating the protein to be modified with a reaction mixture that contains a suitable amount of a galactosyltransferase (galβ1,3 or galβ1,4), a suitable galactosyl donor (e.g., UDP-galactose), a sialyltransferase (α2,3 or α2,6) and a suitable sialic acid donor substrate (e.g., CMP sialic acid). The reaction is allowed to proceed substantially to completion, and then a fucosyltransferase (capable of making an α1,3 or α1,4 linkage) and a suitable fucosyl donor substrate (eg. GDP-fucose). If a fucosyltransferase is used that requires a sialylated substrate (e.g., FucT VII), the reactions can be conducted simultaneously.

a. Sialyltransferase reactions

As discussed above, in some embodiments, the present invention provides a method for fucosylating a glycopeptide following the sialylation of the glycopeptide. In a preferred embodiment, the method produced a population of glycopeptides in which the members have a substantially uniform sialylation pattern. The sialylated glycopeptide is then fucosylation, thereby producing a population of fucosylated peptides in which the members havde a substantially uniform fucosylation pattern.

The method of the invention involves contacting the glycopeptide with a sialyltransferase and a sialic acid donor moiety for a sufficient time and under appropriate reaction conditions to transfer sialic acid from the sialic acid donor moiety to the saccharide groups. Sialyltransferases comprise a family of glycosyltransferases that transfer sialic acid from the donor substrate CMP-sialic acid to acceptor oligosaccharide substrates. In preferred embodiments, the sialyltransferases used in the methods of the invention are recombinantly produced. Suitable sialyltransferase reactions are described in U.S. Provisional Application No. 60/035,710, filed Jan. 16, 1997 and U.S. nonprovisional Application No. 09/007,741, filed Jan. 15, 1998.

Typically, the saccharide chains on a glycopeptide having sialylation patterns altered by the methods of the invention will have a greater percentage of terminal galactose residues sialylated than the unaltered glycopeptide. Preferably, greater than about 80% of terminal galactose residues present on the glycopeptide-linked oligosaccharides will be sialylated following use of the methods. More preferably, the methods of the invention will result in greater than about 90% sialylation, and even more preferably greater than about 95% sialylation of terminal galactose residues. Most preferably, essentially 100% of the terminal galactose residues present on the glycopeptides in the composition are sialylated following modification using the methods of the present invention. The methods are typically capable of achieving the desired level of sialylation in about 48 hours or less, and more preferably in about 24 hours or less.

Examples of recombinant sialyltransferases, including those having deleted anchor domains, as well as methods of producing recombinant sialyltransferases, are found in, for example, U.S. Pat. No. 5,541,083. At least 15 different mammalian sialyltransferases have been documented, and the cDNAs of thirteen of these have been cloned to date (for the systematic nomenclature that is used herein, see, Tsuji et al. (1996) Glycobiology 6: v-xiv). These cDNAs can be used for recombinant production of sialyltransferases, which can then be used in the methods of the invention.

Preferably, for glycosylation of N-linked and/or O-linked carbohydrates of glycopeptides, the sialyltransferase transfer sialic acid to the terminal sequence Galβ1,4-OR or GalNAc-OR, where R is an amino acid, a saccharide, an oligosaccharide or an aglycon group having at least one carbon atom and is linked to or is part of a glycopeptide. Galβ4-GlcNAc is the most common penultimate sequence underlying the terminal sialic acid on fully sialylated carbohydrate structures. At least three of the cloned mammalian sialyltransferases meet this acceptor specificity requirement, and each of these have been demonstrated to transfer sialic acid to N-linked and O-linked carbohydrate groups of glycopeptides. Examples of sialyltransferases that use Galβ-OR as an acceptor are shown in Table 3.

TABLE 3
Mammalian Sialyltransferases

| Sialyltransferase | Sequences formed |
|---|---|
| ST3Ga1 I | Neu5Acα2,3Ga1β1,3Ga1NAc |
| ST3Ga1 II | Neu5Acα2,3Ga1β1,4GlcNAc |
| ST3Ga1 III | Neu5Acα2,3Ga1β1,4GlcNAc |
| | Neu5Acα2,3Ga1β1,3GlcNAc |
| ST3Ga1 IV | Ga1β1,4GlcNAc |
| | Ga1β1,3GlcNAc |
| ST6Ga1NAc I | Neu5Ac2,6Ga1NAc |
| | Ga1β1,3Ga1NAc(Neu5Acα2,6) |
| | Ga1β1,3Ga1NAc(Neu5Acα2,6) |
| | Neu5Acα2,3Ga1β1,3Ga1NAc(Neu5Acα2,6 |
| ST6Ga1NAc II | Neu5Ac2,6Ga1NAc |
| | Ga1β1,3Ga1NAc(Neu5Acα2,6) |

In some embodiments, the invention sialylation methods that have increased commercial practicality through the use of bacterial sialyltransferases, either recombinantly produced or produced in the native bacterial cells. Two bacterial sialyltransferases have been recently reported; an ST6Gal II from Photobacterium damsela (Yamamoto et al. (1996) J. Biochem. 120: 104-110) and an ST3Gal V from Neisseria meningitidis (Gilbert et al. (1996) J. Biol. Chem. 271: 28271-28276). The two recently described bacterial enzymes transfer sialic acid to the Galβ1,4GlcNAc sequence on oligosaccharide substrates. Table 4 shows the acceptor specificity of these and other sialyltransferases useful in the methods of the invention.

TABLE 4
Bacterial Sialyltransferases

| Sialyltransferase | Organism | Structure formed |
|---|---|---|
| Sialyltransferase | N. meningitides | Neu5Acα2,3Ga1β1,4GlcNAc |
| | N. gonorrheae | |
| ST3Ga1 VI | Campylobacter | Neu5Acα2,3Ga1β1,4GlcNAc (also |
| | jejuni | Neu5Acα2,3Ga1β1,3GlcNAc) |
| ST3 Ga1 VII | Haemophilus | |
| | somnus | |
| ST3Ga1 VIII | H. influenzae | |
| ST6Ga1 II | Photobacterium | Neu5Acα2,6Ga1β1,4GlcNAc |
| | damsela | |

A recently reported viral α2,3-sialyltransferase is also suitable for testing and possible use in the sialylation methods of the invention (Sujino et al. (2000) Glycobiology B10: 313-320). This enzyme, v-ST3Gal I, was obtained from Myxoma virus-infected cells and is apparently related to the mammalian ST3Gal IV as indicated by comparison of the respective amino acid sequences. v-ST3Gal I catalyzes the sialylation of Type I (Galβ1,3-GlcNAcβ1-R), Type II (Galβ4GlcNAc-β1-R) and III (Gal β1,3GalNAcβ1-R) acceptors. The enzyme can also transfer sialic acid to fucosylated acceptor moieties (e.g., Lewis^{x} and Lewis^{a}).

An example of a sialyltransferase that is useful in the claimed methods is ST3Gal III, which is also referred to as α(2,3)sialyltransferase (EC 2.4.99.6). This enzyme catalyzes the transfer of sialic acid to the Gal of a Galβ1,3GlcNAc Galβ1,3GalNAc or Galβ1,4GlcNAc glycoside (see, e.g., Wen et al. (1992) J. Biol. Chem. 267: 21011; Van den Eijnden et al. (1991) J. Biol Chem. 256: 3159). The sialic acid is linked to a Gal with the formation of an α-linkage between the two saccharides. Bonding (linkage) between the saccharides is between the 2-position of NeuAc and the 3-position of Gal. This particular enzyme can be isolated from rat liver (Weinstein et al. (1982) J. Biol. Chem. 257: 13845); the human cDNA (Sasaki et al. (1993) J. Biol. Chem. 268: 22782-22787; Kitagawa & Paulson (1994) J. Biol. Chem. 269: 1394-1401) and genomic (Kitagawa et al. (1996) J. Biol. Chem. 271: 931-938) DNA sequences are known, facilitating production of this enzyme by recombinant expression. In a preferred embodiment, the claimed sialylation methods use a rat ST3Gal III.

Other sialyltransferases, including those listed above, are also useful in an economic and efficient large scale process for sialylation of commercially important glycopeptides. As described above, a simple test to find out the utility of these other enzymes, is to react various amounts of each enzyme (1-100 mU/mg protein) with a readily available glycopeptide protein such as asialo-α₁-AGP (at 1-10 mg/ml) to compare the ability of the sialyltransferase of interest to sialylate glycopeptides. The results can be compared to, for example, either or both of an ST6Gal I or an ST3Gal III (e.g., a bovine or human enzyme), depending upon the particular sialic acid linkage that is desired. Alternatively, other glycopeptides or glycopeptides, or N- or O-linked oligosaccharides enzymatically released from the peptide backbone can be used in place of asialo-α₁ AGP for this evaluation, or one can use saccharides that are produced by other methods or purified from natural products such as milk. Preferably, however, the sialyltransferases are assayed using an oligosaccharide that is linked to a glycopeptide. Sialyltransferases showing an ability to, for example, sialylate N-linked or O-linked oligosaccharides of glycopeptides more efficiently than ST6Gal I are useful in a practical large scale process for glycopeptide sialylation.

The invention also provides methods of altering the sialylation pattern of a glycopeptide prior to fucosylation by adding sialic acid in an α2,6Gal linkage as well as the α2,3Gal linkage, both of which are found on N-linked oligosaccharides of human plasma glycopeptides. In this embodiment, ST3Gal III and ST6Gal I sialyltransferases are both present in the reaction and provide proteins having a reproducible ratio of the two linkages formed in the resialylation reaction. Thus, a mixture of the two enzymes may be of value if both linkages are desired in the final product.

An acceptor moiety for the sialyltransferase is present on the glycopeptide to be modified by the sialylation methods described herein. Suitable acceptors include, for example, galactosylated acceptors such as Galβ1,4GlcNAc, Galβ1,4GalNAc, Galβ1,3GalNAc, Galβ1,3GlcNAc, Galβ1,3Ara, Galβ1,6GlcNAc, Galβ1,4Glc (lactose), GalNAc-O-Ser, GalNAc-O-Thr, and other acceptors known to those of skill in the art (see, e.g., Paulson et al. (1978) J. Biol. Chem. 253: 5617-5624). Typically, the acceptors are included in oligosaccharide chains that are attached to asparagine, serine, or threonine residues present in a protein.

B. Glycosyltransferase Reaction Mixtures

The glycosyltransferases, glycopeptides, and other reaction mixture ingredients described above are combined by admixture in an aqueous reaction medium (solution). The medium generally has a pH value of about 5 to about 8.5. The selection of a medium is based on the ability of the medium to maintain pH value at the desired level. Thus, in some embodiments, the medium is buffered to a pH value of about 7.5. If a buffer is not used, the pH of the medium should be maintained at about 5 to 8.5, depending upon the particular glycosyltransferase used. For fucosyltransferases, the pH range is preferably maintained from about 7.2 to 7.8. For sialyltransferases, the range is preferably from about 5.5 and about 6.5. A suitable base is NaOH, preferably 6 M NaOH.

Enzyme amounts or concentrations are expressed in activity Units, which is a measure of the initial rate of catalysis. One activity Unit catalyzes the formation of 1 μmol of product per minute at a given temperature (typically 37° C.) and pH value (typically 7.5). Thus, 10 Units of an enzyme is a catalytic amount of that enzyme where 10 μmol of substrate are converted to 10 μmol of product in one minute at a temperature of 37° C. and a pH value of 7.5.

The reaction mixture may include divalent metal cations (Mg²⁺, Mn²⁺). The reaction medium may also comprise solubilizing detergents (e.g., Triton or SDS) and organic solvents such as methanol or ethanol, if necessary. The enzymes can be utilized free in solution or can be bound to a support such as a polymer. The reaction mixture is thus substantially homogeneous at the beginning, although some precipitate can form during the reaction.

The temperature at which an above process is carried out can range from just above freezing to the temperature at which the most sensitive enzyme denatures. That temperature range is preferably about 0° C. to about 45° C., and more preferably at about 20° C. to about 37° C.

The reaction mixture so formed is maintained for a period of time sufficient to obtain the desired high yield of desired oligosaccharide determinants present on oligosaccharide groups attached to the glycopeptide to be glycosylated. For large-scale preparations, the reaction will often be allowed to proceed for about 8-240 hours, with a time of between about 12 and 72 hours being more typical.

In embodiments in which more than one glycosyltransferase is used to obtain the compositions of glycopeptides having substantially uniform glycopeptides, the enzymes and reagents for a second glycosyltransferase reaction can be added to the reaction medium once the first glycosyltransferase reaction has neared completion. For some combinations of enzymes, the glycosyltransferases and corresponding substrates can be combined in a single initial reaction mixture; the enzymes in such simultaneous reactions preferably do not form a product that cannot serve as an acceptor for the other enzyme. For example, most sialyltransferases do not sialylate a fucosylated acceptor, so unless a fucosyltransferase that only works on sialylated acceptors is used (e.g., FucT VII), a simultaneous reaction by both enzymes will most likely not result in the desired high yield of the desired oligosaccharide determinant. By conducting two glycosyltransferase reactions in sequence in a single vessel, overall yields are improved over procedures in which an intermediate species is isolated. Moreover, cleanup and disposal of extra solvents and by-products is reduced.

One or more of the glycosyltransferase reactions can be carried out as part of a glycosyltransferase cycle. Preferred conditions and descriptions of glycosyltransferase cycles have been described. A number of glycosyltransferase cycles (for example, sialyltransferase cycles, galactosyltransferase cycles, and fucosyltransferase cycles) are described in U.S. Pat. No. 5,374,541 and WO 9425615 A. Other glycosyltransferase cycles are described in Ichikawa et al. J. Am. Chem. Soc. 114:9283 (1992), Wong et al. J. Org. Chem. 57: 4343 (1992), DeLuca, et al., J. Am. Chem. Soc. 117:5869-5870 (1995), and Ichikawa et al. In Carbohydrates and Carbohydrate Polymers. Yaltami, ed. (ATL Press, 1993).

Other glycosyltransferases can be substituted into similar transferase cycles as have been described in detail for the fucosyltransferases and sialyltransferases. In particular, the glycosyltransferase can also be, for instance, glucosyltransferases, e.g., Alg8 (Stagljov et al., Proc. Natl. Acad. Sci. USA 91:5977 (1994)) or Alg5 (Heesen et al. Eur. J. Biochem. 224:71 (1994)), N-acetylgalactosaminyltransferases such as, for example, α(1,3) N-acetylgalactosaminyltransferase, β(1,4) N-acetylgalactosaminyltransferases (Nagata et al. J. Biol. Chem. 267:12082-12089 (1992) and Smith et al. J. Biol Chem. 269:15162 (1994)) and polypeptide N-acetylgalactosaminyltransferase (Homa et al. J. Biol Chem. 268:12609 (1993)). Suitable N-acetylglucosaminyltransferases include GnTI (2.4.1.101, Hull et al., BBRC 176:608 (1991)), GnTII, and GnTIII (Ihara et al. J. Biochem. 113:692 (1993)), GnTV (Shoreiban et al. J. Biol. Chem. 268: 15381 (1993)), O-linked N-acetylglucosaminyltransferase (Bierhuizen et al. Proc. Natl. Acad. Sci. USA 89:9326 (1992)), N-acetylglucosamine-1-phosphate transferase (Rajput et al. Biochem J. 285:985 (1992), and hyaluronan synthase. Suitable mannosyltransferases include α(1,2) mannosyltransferase, α(1,3) mannosyltransferase, β(1,4) mannosyltransferase, Dol-P-Man synthase, OCh1, and Pmt1.

For the above glycosyltransferase cycles, the concentrations or amounts of the various reactants used in the processes depend upon numerous factors including reaction conditions such as temperature and pH value, and the choice and amount of acceptor saccharides to be glycosylated. Because the glycosylation process permits regeneration of activating nucleotides, activated donor sugars and scavenging of produced PPi in the presence of catalytic amounts of the enzymes, the process is limited by the concentrations or amounts of the stoichiometric substrates discussed before. The upper limit for the concentrations of reactants that can be used in accordance with the method of the present invention is determined by the solubility of such reactants.

Preferably, the concentrations of activating nucleotides, phosphate donor, the donor sugar and enzymes are selected such that glycosylation proceeds until the acceptor is consumed. The considerations discussed below, while in the context of a sialyltransferase, are generally applicable to other glycosyltransferase cycles.

Each of the enzymes is present in a catalytic amount. The catalytic amount of a particular enzyme varies according to the concentration of that enzyme's substrate as well as to reaction conditions such as temperature, time and pH value. Means for determining the catalytic amount for a given enzyme under preselected substrate concentrations and reaction conditions are well known to those of skill in the art.

C. Purification

The products produced by the above processes can be used without purification. However, for some applications it is desirable to purify the glycopeptides. Standard, well known techniques for purification of glycopeptides are suitable. Affinity chromatography is one example of a suitable purification method. A ligand that has affinity for a particular glycopeptide or a particular oligosaccharide determinant on a glycopeptide, is attached to a chromatography matrix and the glycopeptide composition is passed through the matrix. After an optional washing step, the glycopeptide is eluted from the matrix.

Filtration can also be used for purification of glycopeptides (see, e.g. U.S. Pat. Nos. 5,259,971 and 6,022,742.

If purification of the glycopeptide is desired, it is preferable that the glycopeptide be recovered in a substantially purified form. However, for some applications, no purification or only an intermediate level of purification of the glycopeptide is required.

The Compositions

In some embodiments, the invention provides a glycopeptide composition that has a substantially uniform glycosylation pattern. The compositions include a saccharide or oligosaccharide that is attached to a protein or glycopeptide for which glycoform alteration is desired. The saccharide or oligosaccharide includes a structure that can function as an acceptor for an enzyme such as a glycosyltransferase, or other enzymes such as a transsialidase, or sulfotransferase. When the acceptor moiety is glycosylated or sulfonated, the desired oligosaccharide structure is formed. The desired structure is one that imparts the desired biological activity upon the glycopeptide to which it is attached. In the compositions of the invention, the preselected saccharide unit is linked to at least about 60% of the potential acceptor moieties of interest. More preferably, the preselected saccharide unit is linked to at least about 80% of the potential acceptor moieties of interest, and still more preferably to at least 95% of the potential acceptor moieties of interest. In situations in which the starting glycopeptide exhibits heterogeneity in the oligosaccharide structure of interest (e.g., some of the oligosaccharides on the starting glycopeptide already have the preselected saccharide unit attached to the acceptor moiety of interest), the recited percentages include such pre-attached saccharide units.

The term “altered” refers to the glycopeptide having a glycosylation pattern that, after application of the methods of the invention, is different from that observed on the glycopeptide as originally produced. For example, the invention provides glycopeptide compositions, and methods of forming such compositions, in which the glycoforms of the glycopeptides are different from those found on the glycopeptide when it is produced by cells of the organism to which the glycopeptide is native. Also provided are compositions, and methods of forming such compositions, in which the glycosylation pattern of a recombinantly produced glycopeptide is modified compared to the glycosylation pattern of the glycopeptide as originally produced by a host cell, which can be of the same or a different species than the cells from which the native glycopeptide is produced.

One can assess differences in glycosylation pattern not only by structural analysis, but also by comparison of one or more biological activities of the protein. A glycopeptide having an “altered glycoform” includes one that exhibits an improvement in one more biological activities of the glycopeptide after the glycosylation reaction compared to the unmodified glycopeptide. For example, an altered glycopeptide includes one that, after glycosylation using the methods of the invention, exhibits a greater binding affinity for a ligand of interest, a greater therapeutic half-life, reduced antigenicity, targeting to specific tissues, and the like. The amount of the improvement observed is preferably statistically significant, and is more preferably at least about a 25% improvement, and still more preferably is at least about 50%, and even still more preferably is at least 80%.

D. Uses for Glycopeptide Compositions

The glycopeptides having desired oligosaccharide determinants described above can then be used in a variety of applications, e.g., as antigens, diagnostic reagents, or as therapeutics. Thus, the present invention also provides pharmaceutical compositions, which can be used in treating a variety of conditions. The pharmaceutical compositions are comprised of glycopeptides made according to the methods described above.

Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249: 1527-1533 (1990).

The pharmaceutical compositions are intended for parenteral, intranasal, topical, oral or local administration, such as by aerosol or transdermally, for prophylactic and/or therapeutic treatment. Commonly, the pharmaceutical compositions are administered parenterally, e.g., intravenously. Thus, the invention provides compositions for parenteral administration which comprise the glycopeptide dissolved or suspended in an acceptable carrier, preferably an aqueous carrier, e.g., water, buffered water, saline, PBS and the like. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

The compositions containing the glycopeptides can be administered for prophylactic and/or therapeutic treatments. In therapeutic applications, compositions are administered to a patient already suffering from a disease, as described above, in an amount sufficient to cure or at least partially arrest the symptoms of the disease and its complications. An amount adequate to accomplish this is defined as a “therapeutically effective dose.” Amounts effective for this use will depend on the severity of the disease and the weight and general state of the patient, but generally range from about 0.5 mg to about 2,000 mg of glycopeptide per day for a 70 kg patient, with dosages of from about 5 mg to about 200 mg of the compounds per day being more commonly used.

In prophylactic applications, compositions containing the glycopeptides of the invention are administered to a patient susceptible to or otherwise at risk of a particular disease. Such an amount is defined to be a “prophylactically effective dose.” In this use, the precise amounts again depend on the patient's state of health and weight, but generally range from about 0.5 mg to about 1,000 mg per 70 kilogram patient, more commonly from about 5 mg to about 200 mg per 70 kg of body weight.

Single or multiple administrations of the compositions can be carried out with dose levels and pattern being selected by the treating physician. In any event, the pharmaceutical formulations should provide a quantity of the glycopeptides of this invention sufficient to effectively treat the patient.

The glycopeptides can also find use as diagnostic reagents. For example, labeled glycopeptides can be used to determine the locations at which the glycopeptide becomes concentrated in the body due to interactions between the desired oligosaccharide determinant and the corresponding ligand. For this use, the compounds can be labeled with appropriate radioisotopes, for example, ¹²⁵I, ¹⁴C, or tritium, or with other labels known to those of skill in the art.

The glycopeptides of the invention can be used as an immunogen for the production of monoclonal or polyclonal antibodies specifically reactive with the compounds of the invention. The multitude of techniques available to those skilled in the art for production and manipulation of various immunoglobulin molecules can be used in the present invention. Antibodies may be produced by a variety of means well known to those of skill in the art.

The production of non-human monoclonal antibodies, e.g., murine, lagomorpha, equine, etc., is well known and may be accomplished by, for example, immunizing the animal with a preparation containing the glycopeptides of the invention. Antibody-producing cells obtained from the immunized animals are immortalized and screened, or screened first for the production of the desired antibody and then immortalized. For a discussion of general procedures of monoclonal antibody production see Harlow and Lane, Antibodies, A Laboratory Manual Cold Spring Harbor Publications, N.Y. (1988).

### EXAMPLES

The present examples exemplify the methods of the invention. Example 1 sets forth the introduction of sialyl Lewis x structures onto a peptide using sialylation and fucosylation in vitro. Example 2 sets forth the results of an investigation into the substrated specificity and fucosylation activity of two fucosyltransferases, FucT-V and FucT-VI. Example 3 sets forth an exemplary fucosylation process of the invention utilizing the protein RsCD4 as a substrate for fucosylation. The fucosylation step is preceded by a sialylation step. Example 4 sets forth an exemplar assay for determining the ability of a fucosyltransferase to act on a particular glycoprotein.

### Example 1

Example 1 sets forth the introduction of sialyl Lewis x structures onto a peptide using using sialylation and fucosylation in vitro.

1.1 Sialylation of Recombinant Glyeopeptide

A glycopeptide was dissolved at 2.5 mg/mL in 50 mM Tris, 0.15M NaCl, 0.05% NaN₃. The solution was incubated with 5 mM CMP-sialic acid and 0.1 U/mL ST3Gal3 at 32° C. for 2 days. To monitor the incorporation of sialic acid, a small aliquot of the reaction had ¹⁴C-CMPSA added; the label incorporated into the peptide was separated from free label by gel filtration on a TosoHaas G2000SWx1 column in 45% MeOH, 0.1% TFA. The radioactivity incorporated into the peptide was quantitated using an in-line scintillation detector. The fraction of label incorporated was found to be 0.073 after 1 day, and 0.071 after two days, indicating that the sialylation reaction was complete in less than 24 hours.

1.2 Fucosylation of the Sialylated Peptide

To the glycopeptide prepared as describe in Example 1.1, GDP-fucose was added to 5 mM, MnCl₂ to 5 mM, and FucT-VI to 0.05 U/mL. The reaction was incubated at 32° C. for 2 days. To monitor incorporation of fucose, a small aliquot of the reaction had ¹⁴C-GDP-fuc added; the label incorporated into the peptide was separated from free label by gel filtration on a TosoHaas G2000SWx1 column in 45% MeOH, 0.1% TFA. The radioactivity was quantitated using an in-line scintillation detector. The fraction of label incorporated was 0.15 after 1 day, and 0.135 after two days, indicating that the fucosylation reaction was complete in less than 24 hours. Following completion of the reaction, N-glycan profiling on FACE gels was carried out according to the GLYKO manual.

1.3 Results

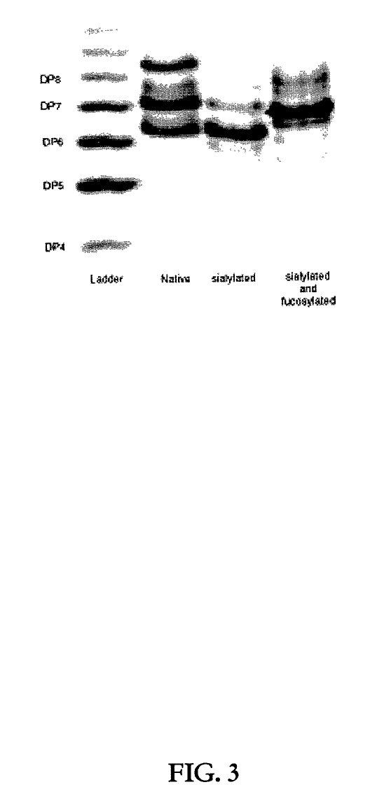

The results of the glycosylation reactions were assayed using FACE analysis. The profile of the N-glycans released from recombinant glycoprotein using PNGase F is provided in FIG. 3. Left to Right: ladder, native; after sialylation with ST3Gal3; after sialylation with ST3Gal3 and fucosylation with FucT-VI. The native material contains a mixture of biantennary, core-fucosylated glycans: asialo (DP 8.5), monosialylated (DP˜7), and disialylated (DP 6.2). After sialylation, the predominant glycan is disialylated (DP6.23). After the fucosylation reaction, there is near quantitative conversion to a band of DP 6.88.

### Example 2

Example 2 sets forth the results of an investigation into the substrated specificity and fucosylation activity of two facosyltransferases, FucT-V and FucT-VI.

2.1 Comparison of Fucosylation Using FucT-V and FucT-VI

Sialylated protein from Example 1.1 was dissolved to a concentration of 2.5 mg/mL, and incubated at 32° C. with 5 mM GDP-fucose, 5 mM MnCl₂, 2 mU/mL of alkaline phosphatase, and 0.05 U/mL of either FucT-V or FucT-VI. After an overnight incubation, incorporated fucose was estimated as described above.

2.2 Results

The mole fraction of GDP-fucose incorporated into protein was 0.016 for FTV, and 0.13 for FTVI. Thus, approximately 8-fold more fucose was incorporated using FTVI compared to FTV.

### Example 3

Example 3 sets forth an exemplary fucosylation process of the invention utilizing the protein RsCD4 as a substrate for fucosylation. The fucosylation step is preceded by a sialylation step.

3.1 Sialylation of RsCD4

RsCD4 (2.5 mg/mL) was dissolved in 25 mM Na phosphate, 0.15M NaCl, 0.05% NaN₃, and was incubated at 32° C. with 5 mM CMPSA and 0.1 U/mL ST3Gal3 for 2 days. After dialysis to remove CMPSA, an aliquot was subjected to N-glycan profiling by FACE according to the GLYKO protocol.

3.2 Results of Sialylation

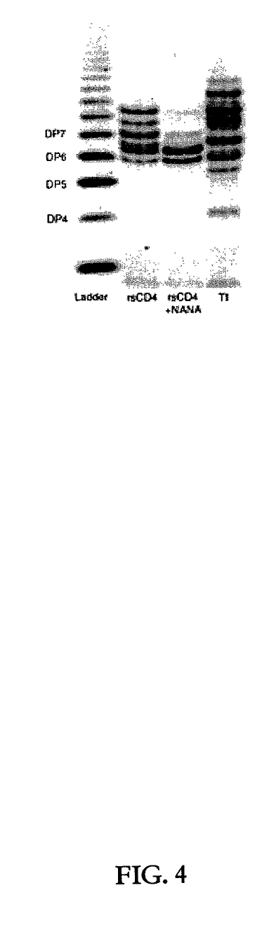

The results of the sialylation reaction are set forth in FIG. 4. In FIG. 4, the native material contains a variety of glycoforms corresponding to bi-antennary glycans with 0,1, or two sialic acids, with and without core fucose. After sialylation, the predominant band is at DP 6.2, which corresponds to a core-fucosylated, disialylated, bi-antennary glycan. The lower band (DP˜5.9) is a non-core fucosylated, disialylated bi-antennary glycan.

3.3 Fucosylation of Sialylated Product

Sialylated rsCD4 (2 mg/mL) from Example 3.1 was dialyzed into 0.1 M Tris, pH 7.2, containing 0.05% NaN₃. The resulting solution was incubated at 32° C. with 5 mM GDP-fucose, 5 mM MnCl₂, and 0.04 U/mL FTVI for two days. After dialysis to remove GDP-fucose, an aliquot was subjected to N-glycan profiling by FACE according to the GLYKO protocol.

3.4 Results

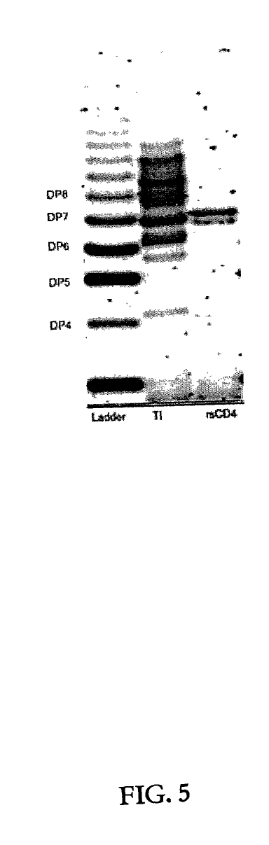

The FACE gel of the product from Example 3.3 is provided in FIG. 5. In FIG. 5, the doublet of bands at DP 5.9 and 6.2 shift after fucosylation with FucT-VI to a doublet at 6.82 and 7.15, indicating the addition of one or more fucose residues.

### Example 4

Example 4 sets forth an exemplar assay for determining the ability of a fucosyltransferase to act on a particular glycoprotein.

Target glycoprotein (1-5 mg/mL) in a suitable buffer (e.g., Tris-buffered saline, pH 7.2) is incubated with 5 mM CMPSA and ST3Gal3 (0.02 U/mg glycoprotein) at 32° for 1 day to fully sialylate potential acceptor sites. GDP-fucose is then added to 5 mM, MnCl₂ to 5 mM, and the appropriate fucosyltransferase (0.02 U/mg glycoprotein) added, along with a tracer amount of radiolabeled GDP-fucose. After 24 h, the amount of radiolabeled fucose incorporated into protein is determined by separating incorporated label from unincorporated label by gel filtration on a TosoHaas G2000SWx1 column in 45% MeOH, 0.1% TFA. Radioactivity is quantified by using an in-line scintillation detector or by collecting fractions, adding scintillant, and using a scintillation counter. The fraction of label incorporated (cpm associated with protein/total cpm) can then be calculated for each fucosyltransferase.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method for modifying the glycosylation pattern of a glycopeptide comprising an acceptor moiety for a first fucosyltransferase, said method comprising:
contacting the glycopeptide with a reaction mixture that comprises a facose donor moiety and the first fucosyltransferase under appropriate conditions to transfer fucose from the fucose donor moiety to the acceptor moiety, such that the glycopeptide has a substantially uniform fucosylation pattern.

2. The method according to claim 1, wherein the glycopeptide comprises a second acceptor moiety for a second fucosyltransferase, and the method further comprises contacting the glycopeptide with a reaction mixture that comprises a fucose donor moiety and the second fucosyltransferase under appropriate conditions to transfer fucose from the fucose donor moiety to the acceptor moiety, such that the glycopeptide has a substantially uniform fucosylation pattern.

3. The method according to claim 2, wherein the glycoprotein is contacted with the first fucosyltranferase and the second fucosyltransferase simultaneously.

4. The method according to claim 2, wherein the glycoprotein is contacted with the first fucosyltransferase and the second fucosyltransferase sequentially without isolation of product resulting from contacting with the first fucosyltransferase.

5. The method according to claim 1, wherein the first fucosyltransferase is a member selected from FucT-IV, FucT-VI, FucT-VII and combinations thereof.

6. The method according to claim 2, wherein the second fucosyltransferase is a member selected from FucT-IV, FucT-VI, FucT-VII and combinations thereof.

7. The method of claim 1, wherein the fucosyltransferase is bacterial.

8. The method of claim 1, wherein the fucosyltransferase is recombinantly produced.

9. The method of claim 1, wherein the fucosyltransferase lacks a membrane anchoring domain.

10. The method of claim 1, wherein at least about 80% of the acceptor moieties on the glycopeptide are fucosylated.

11. The method of claim 1, wherein glycopeptide is reversibly immobilized on a solid support.

12. The method of claim 1, wherein the solid support is an affinity chromatography medium.

13. The method of claim 1, wherein the glycopeptide is a full-length glycopeptide.

14. The method of claim 1, wherein the glycopeptide is a fragment of a full length glycopeptide comprising an active site of the full-length glycopeptide.

15. The method according claim 1, wherein the glycopeptide is an IgG chimera.

16. The method of claim 1, wherein the glycopeptide is a hormone, a growth factor, an enzyme, an enzyme inhibitor, a cytokine, a receptor, a ligand, or a monoclonal antibody.

17. The method of claim 1, wherein the glycopeptide is on a cell.

18. The method of claim 1, wherein the acceptor moiety comprises Galβ1-OR, Galβ1,3/4GlcNAc-OR, NeuAcα2,3Galβ1,3/4GlcNAc-OR, wherein R is an amino acid, a saccharide, an oligosaccharide or an aglycon group having at least one carbon atom and is linked to or is part of a glycopeptide.

19. The method of claim 1, wherein the fucose donor moiety is GDP-fucose.

20. The method of claim 1, further comprising, prior to step (a), contacting said glycoprotein with a glycosyltransferase other than a fucosyltransferase and a donor moiety other than a fucose donor moiety, thereby glycosylating the glycoprotein with a glycosyl moiety other than a fucose unit.

21. The method of claim 20, wherein the glycosyltransferase is a member selected from the group consisting of galactosyltransferase, sialyltransferase and combinations thereof.

22. A composition comprising a glycopeptide fucosylated according to the method of claim 1.

23. The composition of claim 22, wherein at least 80% of the acceptor moieties on the glycopeptide are fucosylated.

24. The composition of claim 22, wherein glycopeptide is attached to a solid support.

25. The composition of claim 24, wherein the solid support is an affinity chromatography medium.

26. The composition of claim 22, wherein the glycopeptide is a full-length glycopeptide.

27. The composition of claim 22, wherein the glycopeptide comprises Fucα1,2Galβ1-OR, Galβ1,3/4(Fucα1,4/3)GlcNAc-OR, NeuAcα2,3 Galβ1,3/4(Fucα1,3/4)GlcNAc-OR, Fucα1,2Galβ1,3/4(Fucα1,4/3)GlcNAcβ-OR wherein R is an amino acid, a saccharide, an oligosaccharide or an aglycon group having at least one carbon atom and is linked to or is part of a glycopeptide.

28. The, composition of claim 22, wherein the glycopeptide comprises NeuAcα2,3Galβ1,3/4(Fucα1,3/4)GlcNAc-OR, wherein R is an amino acid, a saccharide, an oligosaccharide or an aglycon group having at least one carbon atom and is linked to or is part of a glycopeptide.

29. The composition of claim 22, wherein the glycopeptide is a hormone, a growth factor, an enzyme, an enzyme inhibitor, a cytokine, a receptor, a ligand, or a monoclonal antibody.

30. The composition of claim 22, wherein the glycopeptide is on a cell.

31. A method of producing a recombinant glycopeptide having a fucosylation pattern that is substantially identical to a fucosylated glycopeptide having a known fucosylation pattern, said method comprising:
(a) contacting the recombinant glycopeptide with a reaction mixture that comprises a fucose donor moiety and the fucosyltransferase under appropriate conditions to transfer fucose from the fucose donor moiety to a fucose acceptor moiety on said recombinant glycopeptide, thereby producing a fucosylated recombinant glycopeptide; and
(b) terminating the transfer of the fucose to the fucose acceptor when the fucosylation pattern substantially identical to the known fucosylation pattern is obtained.

32. The method according to claim 31 further comprising:
(c) assaying the fucosylation pattern of the fucosylated recombinant glycopeptide, thereby determining whether the fucosylation pattern is substantially identical to the known fucosylation pattern.

33. The method according to claim 31 wherein the terminating is due to exhausting in the reaction mixture a member selected from the group consisting of the fucosyltransferase, the fucose donor moiety, the fucose acceptor quench with a chelator and combinations thereof.

34. The method according to claim 31, wherein the glycopeptide comprises a second acceptor moiety for a second fucosyltransferase, and the method further comprises contacting the glycopeptide with a reaction mixture that comprises a fucose donor moiety and the second fucosyltransferase under appropriate conditions to transfer fucose from the fucose donor moiety to the second acceptor moiety.

35. The method according to claim 34, wherein the glycoprotein is contacted with the first fucosyltranferase and the second fucosyltransferase simultaneously.

36. The method according to claim 34, wherein the glycoprotein is contacted with the first fucosyltransferase and the second fucosyltransferase sequentially without isolation of product resulting from contacting with the first fucosyltransferase.

37. The method according to claim 31, wherein the first fucosyltransferase is a member selected from FucT-IV, FucT-VI, FucT-VII and combinations thereof.

38. The method according to claim 34, wherein the second fucosyltransferase is a member selected from FucT-IV, FucT-VI, FucT-VII and combinations thereof.

39. The method of claim 31, wherein the fucosyltransferase is bacterial.

40. The method of claim 31, wherein the fucosyltransferase is recombinantly produced.

41. The method of claim 31, wherein the fucosyltransferase lacks a membrane anchoring domain.

42. The method of claim 31, wherein at least about 80% of the acceptor moieties on the glycopeptide are fucosylated.

43. The method of claim 31, wherein glycopeptide is reversibly immobilized on a solid support.

44. The method of claim 31, wherein the solid support is an affinity chromatography medium.

45. The method of claim 31, wherein the glycopeptide is a full-length glycopeptide.

46. The method of claim 31, wherein the glycopeptide is a fragment of a full length glycopeptide comprising an active site of the full-length glycopeptide.

47. The method according claim 31, wherein the glycopeptide is an IgG chimera.

48. The method of claim 31, wherein the glycopeptide is a hormone, a growth factor, an enzyme, an enzyme inhibitor, a cytokine, a receptor, a ligand, or a monoclonal antibody.

49. The method of claim 31 wherein the glycopeptide is on a cell.

50. The method of claim 31, wherein the acceptor moiety comprises Galβ1-OR, Galβ1,3/4GlcNAc-OR, NeuAcα2,3Galβ1,3/4GlcNAc-OR, wherein R is an amino acid, a saccharide, an oligosaccharide or an aglycon group having at least one carbon atom and is linked to or is part of a glycopeptide.

51. The method of claim 31, wherein the fucose donor moiety is GDP-fucose.

52. The method of claim 31, further comprising, prior to step (a), contacting said glycoprotein with a glycosyltransferase other than a fucosyltransferase and a donor moiety other than a fucose donor moiety, thereby glycosylating the glycoprotein with a glycosyl moiety other than a fucose unit.

53. The method of claim 52, wherein the glycosyltransferase is a member selected from the group consisting of galactosyltransferase, sialyltransferase and combinations thereof.

54. A large-scale method for modifying the glycosylation pattern of a glycopeptide comprising an acceptor moiety for a first fucosyltransferase, said method comprising:
contacting at least about 500 mg of glycopeptide with a reaction mixture that comprises a fucose donor moiety and the first fucosyltransferase under appropriate conditions to transfer fucose from the fucose donor moiety to the acceptor moiety, such that the glycopeptide has a substantially uniform fucosylation pattern.

55. A large-scale method of producing a recombinant glycopeptide having a fucosylation pattern that is substantially identical to a fucosylated glycopeptide having a known fucosylation pattern, said method comprising:
(a) contacting at least about 500 mg of the the recombinant glycopeptide with a reaction mixture that comprises a fucose donor moiety and the fucosyltransferase under appropriate conditions to transfer fucose from the fucose donor moiety to a fucose acceptor moiety on said recombinant glycopeptide, thereby producing a fucosylated recombinant glycopeptide; and
(b) terminating the transfer of the fucose to the fucose acceptor when the fucosylation pattern substantially identical to the known fucosylation pattern is obtained.
